(19) United States
(12) Patent Application Publication
(10) Pub. No.: US 2024/0286982 A1
(43) Pub. Date: Aug. 29, 2024

(54) PROCESS AND PLANT FOR PRODUCING METHANE OR METHANOL FROM A SOLID RENEWABLE FEEDSTOCK

(71) Applicant: Topsoe A/S, Kgs. Lyngby (DK)

(72) Inventors: Magnus Zingler STUMMANN, Copenhagen OE (DK); Troels Dahlgaard STUMMANN, Copenhagen (DK)

(73) Assignee: Topsoe A/S, Kgs. Lyngby (DK)

(21) Appl. No.: 18/571,911

(22) PCT Filed: Jun. 28, 2022
(86) PCT No.: PCT/EP2022/067734

§ 371 Date: Dec. 19, 2023

(30) Foreign Application Priority Data
Jun. 29, 2021 (EP) 21182476.8

(51) Int. Cl. C07C 29/151; B01J 6/00; B01J 19/24; C01B 3/34; C07C 1/12; C10B 53/00; C10B 53/02; C10L 1/06; C25B 1/042; C25B 15/08

(52) U.S. Cl. CPC C07C 29/1518; B01J 6/008; B01J 19/245; C01B 3/34; C07C 1/12; C10B 53/00; C10B 53/02; C10L 1/06; C25B 1/042; C25B 15/081; C01B 2203/0233; C01B 2203/061; C01B 2203/085; C01B 2203/1235; C01B 2203/1258; C10L 2200/0469; C10L 2200/0492; C10L 2290/02; C10L 2290/42

(57) ABSTRACT

Process for producing methane or methanol, said process comprising the steps of: i) conducting a solid renewable feedstock to a thermal decomposition step, this being a pyrolysis step or a hydrothermal liquefaction step, for producing: a first off-gas stream comprising hydrocarbons, a solid carbon stream, and optionally a first liquid oil stream; upgrading the first off-gas stream by conducting it to a hydro/deoxygenation (HDO/DO) step i.e. hydrodeoxygenation or deoxygenation step in which said HDO/DO step is conducted in the absence of steam, and a subsequent separation step, for generating water, a second liquid oil stream and an upgraded first off-gas stream; ii) conducting the first off-gas stream or the upgraded first off-gas stream to an olefin removal step, for generating a further upgraded first off-gas stream which is free of olefins; iii-1) conducting the first off-gas stream, or the upgraded first off-gas stream, or the further upgraded first off-gas stream, to a methanation step under the generation of steam for producing said methane; or iii-2) conducting the first off-gas stream, or the upgraded first off-gas stream, or the further upgraded first off-gas stream, to a steam reforming step for producing a methanol synthesis gas and subsequently conducting the methanol synthesis gas to a methanol synthesis step under the generation of steam for producing said methanol; iv) conducting steam, such as at least a portion of the steam generated in step iii-1) or iii-2), to an electrolysis step for producing an oxygen stream and a hydrogen stream; v) conducting at least a portion of the hydrogen stream from the electrolysis step to any of the: thermal decomposition step including HDO-step, olefin hydrogenation step, methanation step, methanol synthesis step, or combinations thereof.

### FIELD OF THE INVENTION

The present invention relates to a process and plant for producing valuable products such as methane or methanol from an off-gas derived from the thermal decomposition of a solid renewable feedstock such as from a pyrolysis gas derived from the pyrolysis of a solid renewable feedstock, such as lignocellulosic biomass, and where the pyrolysis gas is optionally upgraded in a hydro/deoxygenation (HDO/DO) step. Embodiments of the invention include converting pyrolysis gas or the upgraded pyrolysis gas into methane or methanol, where the required hydrogen is provided by electrolysis of water (steam) produced in the process. Embodiments of the invention further include optionally separating a first liquid oil stream from the pyrolysis step i.e. a pyrolysis oil (bio-oil) and separating a second liquid oil stream from the upgraded pyrolysis gas. The first and second liquid oil streams are suitably combined and co-fed to a hydroprocessing and subsequent separation step for thereby producing hydrocarbon products in the transportation fuel range such as jet fuel or diesel. The required hydrogen for the process optionally including the hydroprocessing is at least partly provided by electrolysis of water (steam) produced in the process. The methanol may be further converted to gasoline, thus providing an alternative route for producing gasoline from solid renewable feedstocks.

### BACKGROUND OF THE INVENTION

Currently, it is often perceived that there are three generations of renewable feedstocks. The first generation are renewable feedstocks which are already liquid and include virgin oil, rapeseed oil and soybean oil. The second generation are waste oil and fats, such as used cooking oils, animal fats and crude tall oil (CTO). The third generation is much larger in volume, i.e. is more available, than for instance the second generation. This third generation includes solid renewable feedstocks which encompasses: i) solid waste, such as agricultural residue and forestry residue, for instance lignocellulosic biomass such as grass; and ii) low indirect land-use change (ILUC) crops such as castor, which offer the benefit of not competing for space with food crops and can be grown in difficult climates.

Due to i.a. the Renewable Energy Directive II (RED II) under the European Union, a higher demand is expected for the hydroprocessing of advanced renewable feedstocks, such as pyrolysis oils derived from solid renewable feedstocks. A pyrolysis oil may have a very high oxygen content, which needs to be decreased and further treated before it can be used as liquid fuel, i.e. as hydrocarbon fuel boiling in the transportation fuel range, such as diesel. The oxygen is generally removed by hydroprocessing in a catalytic hydrodeoxygenation (HDO) step.

The pyrolysis generates normally a pyrolysis oil and a pyrolysis off-gas which often is flared to the atmosphere.

While lignocellulosic biomass can be converted into liquid fuels (liquid oil) by pyrolysis, approximately 13 wt % of the biomass is converted into gas (light hydrocarbons, CO, and CO₂), which is considered to be an undesirable by-product. In catalytic fast pyrolysis and catalytic fast hydropyrolysis, reactive catalytic fast pyrolysis, and pyrolysis followed by ex-situ HDO i.e. where the pyrolysis gas is sent to a HDO reactor for catalytically deoxygenating it prior to condensation of a pyrolysis oil, the gas yield is generally significantly higher. In particular, when the pyrolysis gas (also referred as pyrolysis vapor) is upgraded in a HDO step, the carbon recovery in the liquid phase is typically only below 50%; for instance, for catalytic hydropyrolysis with the subsequent HDO step the carbon recovery is typically below 50%, while for fast pyrolysis without the subsequent HDO step the carbon recovery is only up to about 70% in the liquid phase.

When conducting pyrolysis, the typical approach is to burn the pyrolysis gas and thereby utilize it to generate the heat used in the pyrolysis process and/or district heating. In catalytic fast hydropyrolysis (catalytic hydropyrolysis), reactive catalytic fast pyrolysis, and pyrolysis followed by ex-situ HDO of the pyrolysis gas/vapor, the resulting light gasses are converted into H₂ in a steam methane reformer. For instance, in refineries for production of hydrocarbon products boiling in the transportation fuel range, it is known to convert light hydrocarbons (C1-C4) produced in the hydroprocessing section of the refinery plant into hydrogen, which can be used in e.g. the HDO step of the hydroprocessing/hydrotreatment section, of the refinery plant.

US 2013/137783 A1 discloses a method and system for converting intermittent renewable energy and renewable carbonaceous feedstock to non-intermittent renewable electrical and thermal energy, storing it as fuels and chemicals and using it to capture and re-use or dispose of CO₂ emissions. Gasification of the renewable carbonaceous feedstock is used to generate gaseous streams from which renewable fuels and renewable chemicals are produced.

GB 2539021 discloses a process for producing a substitute natural gas (SNG), the process comprising the steps of: (1) providing a synthesis gas comprising hydrogen and carbon monoxide; (2) forming a hydrogen-enriched synthesis gas; (3) subjecting the hydrogen-enriched synthesis gas to a methanation reaction to convert at least a portion of the gas into methane thereby forming a methane-enriched gas; and (4) recovering from the methane-enriched gas a methane-containing gas, wherein step (2) comprises providing a hydrogen gas and combining the hydrogen gas with the synthesis gas; and in which the hydrogen may be generated by electrolysis of water. The synthesis gas is provided by gasification.

US 2016/304799 discloses a method for producing hydrocarbons from biomass. The method is particularly useful for producing substitute natural gas from forestry residues. Certain disclosed embodiments convert a biomass feedstock into a product hydrocarbon by fast pyrolysis. The resulting pyrolysis gas is converted to the product hydrocarbon and carbon dioxide in the presence of hydrogen and steam while simultaneously generating the required hydrogen by reaction with steam under prescribed conditions for self-sufficiency of hydrogen. Methane is a preferred hydrocarbon product, and there is no implicit or explicit generation of a liquid oil, so most or nearly all of the carbon in the feed pyrolysis gas ends up in the methane product. Supplemental hydrogen may alternatively be provided as electrolytic hydrogen preferably generated by a renewable energy source such as wind energy, Common in the above citations is the provision of a step such as gasification or hydrogasification, typically requiring temperatures of 650° C. or higher, for producing a gaseous stream—a synthesis gas—for further conversion into e.g. substitute natural gas (SNG), thereby providing a synthesis gas stream representing at least 90 wt % of the renewable feed. The citations are silent on the provision of a liquid oil stream from which valuable hydrocarbon products may be generated, where most of the carbon in e.g. pyrolysis off-gas is recovered in the liquid phase, not in the gas phase; and much less so on how to advantageously integrate the production of methane (SNG) or methanol, together with the production of hydrocarbons in the transportation fuel range, such as jet fuel, diesel and maritime (marine) fuel.

### SUMMARY OF THE INVENTION

It is an object of the present invention to provide a process and plant with increased carbon recovery of a solid renewable feedstock when upgrading pyrolysis gas generated from the pyrolysis of the feedstock.

It is another object of the present invention to increase the carbon recovery while at the same time producing valuable products such as methane and methanol.

It is another object of the present invention to be able to provide an alternative route for the generation of the full range of transportation fuels including diesel, jet fuel and gasoline, all derived from the same original solid renewable feedstock.

It is a further object of the present invention to provide high integration of the process/plant for producing methane or methanol from a pyrolysis gas and a pyrolysis solid carbon stream.

It is yet a further object of the present invention to provide high integration of the process/plant for producing methane or methanol from a pyrolysis gas and a pyrolysis solid carbon stream, with a refinery process/plant comprising a hydroprocessing and separation step for treating pyrolysis oil(s) generated in the process for producing methane or methanol.

These and other objects are solved by the present invention.

Accordingly, in a first aspect the invention is a process for producing methane or methanol, said process comprising the steps of:
i) conducting a solid renewable feedstock to a thermal decomposition step for producing: a first off-gas stream comprising hydrocarbons, a solid carbon stream i.e. char, and optionally a first liquid oil stream; wherein the thermal decomposition step is a pyrolysis step or a hydrothermal liquefaction step;
upgrading the first off-gas stream by conducting it to a hydro/deoxygenation (HDO/DO) step i.e. hydrodeoxygenation (HDO) or deoxygenation (DO) step, in which said HDO/DO step is conducted in the absence of steam, and a subsequent separation step for generating water, a second liquid oil stream and an upgraded first off-gas stream;
ii) conducting the upgraded first off-gas stream to an olefin removal step, for generating a further upgraded first off-gas stream which is free of olefins;
iii-1) conducting the further upgraded first off-gas stream to a methanation step under the generation of steam for producing said methane;
or
iii-2) conducting the further upgraded first off-gas stream to a steam reforming step for producing a methanol synthesis gas and subsequently conducting the methanol synthesis gas to a methanol synthesis step under the generation of steam for producing said methanol;
iv) conducting steam, such as at least a portion of the steam generated in step iii-1) or iii-2), to an electrolysis step for producing an oxygen stream and a hydrogen stream;
v) conducting at least a portion of the hydrogen stream from the electrolysis step to any of the: thermal decomposition step including the HDO-step, olefin hydrogenation step, methanation step, methanol synthesis step, or combinations thereof.

It has been found, that by incorporating methanation, or methanol synthesis with associated prior steam reforming, the carbon recovery over the optional HDO/DO step and downstream methanation, or over the optional HDO/DO and downstream steam reforming and methanol synthesis, is increased to near 100%. Hence, all carbon in the solid renewable stock, apart from the solid carbon stream (char) withdrawn in step i) in connection with the thermal decomposition, is advantageously converted into the valuable products methane or methanol, as well as a liquid oil, e.g. the second liquid oil stream, from which valuable hydrocarbon products may be generated, such as hydrocarbon products in the transportation fuel range, e.g. naphtha, jet fuel, diesel, and maritime (marine) fuel.

In an embodiment, the process further comprises:
i-1) combusting at least a portion of the solid carbon stream with at least a portion of the oxygen stream from the electrolysis, for generating heat and a carbon dioxide stream; and conducting at least a portion of the carbon dioxide stream to step iii-1) or iii-2);
or
i-2) conducting at least a portion of the solid carbon stream to a gasification step with steam and/or oxygen produced in the process, such as at least a portion of the steam generated in step iii-1) and/or at least a portion of the oxygen stream from electrolysis; optionally with indirect heating; for producing a synthesis gas stream and conducting it to step iii-1) or iii-2).

Thereby, in step i-1) carbon dioxide is utilized as a valuable feed source for methane or methanol production. The carbon in the char is also recovered and thus all carbon in the solid renewable stock is advantageously converted into the valuable products methane or methanol. By using the O₂ rich off gas from the electrolysis (oxygen stream from the electrolysis, containing for instance about 40 vol. % O₂) for the combustion of the char, the CO₂ gas will be lean in N₂, which is desirable for the methanation step or methanol synthesis step, as N₂ is an unwanted inert.

In step i-2) the char is fed to a gasification step with steam (H₂O) and/or O₂, suitably with steam generated in the process e.g. in step iv-1) or iv-2) and/or oxygen produced in the process, more specifically being produced in the electrolysis step. Indirect heating is applied, such as by the burning of a hydrocarbon rich off gas, e.g. a Pressure Swing Adsorption off-gas (PSA off gas) or a methanol synthesis purge gas, a flash gas from an amine absorption unit, or other gases with a too high inert level and/or a too low quality for further recovery which are generated in the process. For instance, methanol synthesis purge gas is advantageously used as fuel gas in order to avoid recycle of inert gases such as N₂. Thereby, a synthesis gas (additional synthesis gas) is produced which is then used in the methanation (iii-1) or methanol synthesis step (iii-2) and thus is also advantageously converted into the valuable products methane or methanol.

It would be understood, that the term “hydro/deoxygenation (HDO/DO) step” denotes: hydrodeoxygenation (HDO) whereby hydrogen is added; or deoxygenation (DO) whereby no hydrogen is added.

It would be understood, that throughout this application the term “at least a portion” of a given stream means a portion thereof, or the entire stream.

It would be understood that the term “pyrolysis solid carbon stream” is used interchangeably with the term “solid carbon stream” or the term “char” where the thermal decomposition step is a pyrolysis step.

It would be understood that the term “further upgraded first off-gas stream which is free of olefins” denotes a stream after the olefin removal step iii) and having 5 wt % or less of olefin compounds.

The term “comprising” includes also “comprising only”, i.e. “consisting of”.

The term “suitably” means optional, i.e. an optional embodiment.

The terms “present invention” and “present application” are used interchangeably.

The term “first aspect of the invention” means the process according to the invention.

The term “second aspect of the invention” means the plant for conducting the process according to the invention.

Other definitions are provided in connection with one or more of the below embodiments.

Methanation, steam methane reforming and methanol gas synthesis are well-known technologies:

Methanation means the catalytic conversion of a feed gas comprising carbon oxides and hydrogen to methane according to the reactions: CO+3H₂=CH₄+H₂O; CO₂+4 H₂═CH₄+2H₂O.

Steam reforming means the catalytic conversion of a feed gas comprising methane into carbon oxides and hydrogen, i.e. the reverse of the above reactions: CH₄+H₂O═CO+3H₂; CH₄+2H₂O=CO₂+4 H₂.

The steam reforming step may also include pre-reforming i.e. whereby higher hydrocarbons are steam reformed, i.e. according to the reaction (example for ethane): C₂H_{g}+2H₂O→2CO+5H₂. Hence, the prereforming step reduces the concentration of higher hydrocarbons (hydrocarbons with two or more carbon atoms) thereby also reducing the potential for undesired carbon formation in the steam reforming step as well as reducing the inert content in the methanol synthesis.

Methanol synthesis means the catalytic conversion of a methanol synthesis gas into methanol according to the reactions: 3H₂+CO₂=CH₃OH+H₂O, CO+2H₂═CH₃OH. The methanol synthesis gas is suitably a synthesis gas having a module M=(H₂—CO₂)/(CO+CO₂) of 1.9-2.1, preferably 2. The synthesis gas used for methanol production, i.e. methanol synthesis gas, is normally described in terms of said module M, since the synthesis gas is in balance for the methanol reaction when M=2. In typical synthesis gases for methanol production, such as methanol synthesis gas produced by steam reforming, the synthesis gas will contain some excess hydrogen resulting in modules slightly above 2, for instance 2.05 or 2.1. This methanol synthesis gas is then passed to a conventional methanol loop including conversion into methanol (CH₃OH) in a methanol synthesis reactor according to the above reactions. The resulting raw methanol stream is then purified, i.e. enriched in methanol, e.g. via distillation, thereby producing a product stream with at least 98 wt % methanol as well as a separate water stream.

Methanol technology including methanol synthesis reactors and/or methanol synthesis loops are well-known in the art. Thus, the general practice in the art is conducting the methanol conversion in a once-through methanol conversion process; or to recycle unconverted synthesis gas separated from the reaction effluent and dilute the fresh synthesis gas with the recycle gas. The latter typically results in the so-called methanol synthesis loop with one or more reactors connected in series or in parallel. For instance, serial synthesis of methanol is disclosed in U.S. Pat. Nos. 5,827,901 and 6,433,029, and parallel synthesis in U.S. Pat. No. 5,631,302 and EP 2874738 E1.

In an embodiment, the process further comprises converting the methanol into gasoline. Thereby a simple alternative route for the production of a hydrocarbon boiling in the transportation fuel range, here gasoline, from the thermal decomposition of a solid renewable feedstock is possible. For producing gasoline, the conventional route would otherwise normally be by separately hydroprocessing the first liquid oil stream.

The conversion of methanol to gasoline is well-known in the art, for instance by applicant's TIGAS™ process (methanol to gasoline process). See also e.g. U.S. Pat. Nos. 4,788,369, 4,481,305 or 4,520,216. In the methanol to gasoline process, a raw gasoline product is produced, which prior to further upgrading into said gasoline (gasoline product) is rich in monoaromatics, such as at least 50 wt %, and typically above 80 wt % but contains only few normal-paraffins (less than 1 wt %), a moderate amount of iso-paraffins (such as 5 wt % to 10 wt %), few olefins (less than 5 wt %) and is virtually free of di-olefins (less than 0.5 wt %). The raw gasoline product is further characterized by the C9 aromatics being dominated by 1,2,4-trimethyl benzene (the concentration of 1,2,4-trimethylbenzene is above 2 wt % and the ratio of 1,2,4-trimethyl benzene to 1,2,3-trimethyl benzene is above 6 and typically above 10, contrary to fossil fuel derived gasoline where the ratio is around 4). The raw gasoline product is also characterized by the C10 aromatics being dominated by 1,2,4,5-tetramethyl benzene (the concentration of 1,2,4,5-tetramethyl benzene is above 10 wt % and the ratio to the other tetramethyl benzenes is above 10 and typically above 20, contrary to fossil fuel derived gasoline where 1,2,4,5-tetramethyl benzene is the least common tetramethyl benzene). The compound 1,2,4-trimethyl benzene is also referred to as pseudocumene. The compound 1,2,3-trimethyl benzene is also referred to as hemimellitene. The compound 1,2,4,5-tetramethyl benzene is also referred to as durene.

The first off-gas or upgraded first off-gas from the HDO/DO may contain olefins, for instance C3-C4 olefins. These are removed in the olefin removal step (step ii), suitably by hydrogenation, as is also well known in the art. Due to the olefin content, a gas containing olefins increases the potential of carbon formation thereby causing damage in the catalyst and/or equipment downstream.

Suitably, the olefin removal step is conducted at a temperature of 100-450° C., such as 200-400° C., a pressure of 5-50 bar, and a gas to oil ratio of 2-25 Nm³/m³.

The thermal decomposition step is, in an embodiment, a pyrolysis step, such as a fast pyrolysis step, thereby producing in step i) said first off-gas stream, said solid carbon stream, and optionally said first liquid oil stream, as well as said optional second liquid oil stream and upgraded first off-gas stream. Accordingly, the first off-gas stream is in connection with this embodiment also denoted as a pyrolysis off-gas stream, and the optional first liquid oil stream as a first pyrolysis oil stream. Suitably, the first pyrolysis oil stream is produced by condensing it from the first pyrolysis off-gas exiting the pyrolysis unit and prior to conducting the pyrolysis off-gas to the HDO step. Apart from the first pyrolysis off-gas stream and the first pyrolysis oil stream, the solid carbon stream (char) is also generated and withdrawn from the process.

By conducting a pyrolysis or hydrothermal liquefaction step, e.g. at 300-600° C., followed by HDO/DO as in the present application, liquids or gas-liquid intermediate products are further converted to a liquid oil and further to hydrocarbon products, such as liquid hydrocarbons in the transportation fuel range. Thus, the upgraded first off-gas stream is less than 80 wt % of the solid renewable feedstock. This is in stark contrast with prior art processes, where gasification or similar such as hydrogasification, typically conducted at 650° C. or higher, is performed, and which results in synthesis gas (syngas) as the major intermediate product, typically 90 wt % or more of the feedstock ends up in the gas; or in a feed pyrolysis gas of which most or nearly all of the carbon ends up in a gaseous methane-rich product.

Accordingly, in the present invention, the thermal decomposition step is not gasification, i.e. there is no gasification unit to provide a first off-gas stream comprising hydrocarbons, a solid carbon stream, and optionally a first liquid oil stream. Furthermore, the upgraded first off-gas stream is less than 80 wt % of said solid renewable feedstock.

By the present invention, the carbon recovery in the liquid phase with respect to the first off-gas stream is 60 wt % or more, such as 70 wt % or more, e.g. in the range 60-90 wt %. Accordingly, in an embodiment, of the first off-gas stream (pyrolysis off-gas fed to HDO unit), a minor portion of the carbon, i.e. 40 wt % or less, such as 35 wt % or less, e.g. e.g. 10 wt %, 20 wt %, 25 wt %, or 30 wt %, for instance 20-40 wt % or 25-35 wt %, ends up in the methane or methanol; and 60 wt % or more, such as 65 wt % or more, e.g. 70 wt %, e.g. 75 wt % or 80 wt % or 90 wt %, for instance 60-80 wt % or 65-75 wt %, ends up in the second liquid oil stream. For instance, 10-40 wt % of the carbon of the first off-gas stream ends up in the methane or methanol, and 60-90 wt % ends up in the second liquid oil stream.

The second liquid oil stream is highly useful and advantageous for production of hydrocarbon products, as it will also become apparent from one or more of below embodiments.

It would be understood, that the term “first liquid oil stream” represents, in a particular embodiment e.g. when conducting simple fast pyrolysis as described farther below, the liquid oil withdrawn prior to the HDO step in step i), said first liquid oil stream thus being associated with the thermal decomposition step, while the term “second liquid oil stream” represents the liquid oil withdrawn after conducting the HDO step, more specifically in the subsequent separation step therein. It would also be understood that by HDO, under the addition of hydrogen, oxygen is catalytically removed as water. Accordingly, in the subsequent separation step, a water stream is also withdrawn. HDO may also comprise decarboxylation whereby oxygen is removed as CO or CO₂, as also known in the art.

The HDO/DO step is conducted in the absence of steam. Thus, contrary to the prior art such as US 2016304799 A1, where hydrogasification and thus presence of steam is needed to generate methane in the gas, the present application provides most of the hydrocarbons in the liquid phase instead of in the gas. By the present application, as recited above, the upgraded first off-gas stream is less than 80 wt % of said solid renewable feedstock, whereas in gasification, such as hydrogasification, more than 90 wt % of a solid renewable feedstock will end up in the first off-gas stream. Further, in the HDO/DO step, typically, zeolite catalysts are utilized, as so are the supports (carriers), for which the presence of steam promotes oxidation conditions, which is undesirable. The HDO/DO step in the absence of steam according to the present application promotes instead reducing conditions.

According to the present invention, when processing a solid renewable feedstock —suitably represented as [CH₂O]ₓ— into oil—suitably represented as [CH₂]ₓ—, methane (CH₄) or methanol (CH₃OH), there is a deficit of H₂, and which is required for removing oxygen in the feedstock as H₂O. The balance between H and C is established in three ways:
removing C as solid carbon
removing C as CO₂
adding H₂ from a separate source like electrolysis

The pyrolysis step may include the use of a fluidized bed, transported bed, or circulating fluid bed, as is well known in the art. For instance, the pyrolysis step may comprise the use of a pyrolysis unit (also denoted as pyrolysis reactor), cyclone(s) to remove particulate solids such as char, and a cooling unit for thereby producing said first off-gas stream (i.e. pyrolysis off-gas) and said first liquid oil stream, i.e. condensed pyrolysis oil. This first off-gas stream comprises light hydrocarbons e.g. C1-C4 hydrocarbons, CO and CO₂. The first liquid oil stream is also referred to as pyrolysis oil or bio-oil and is a liquid substance rich in blends of molecules usually consisting of more than two hundred different compounds including aldehydes, ketones and/or other compounds such as furfural having a carbonyl group, resulting from the depolymerisation of products treated in pyrolysis.

While by the present invention a first liquid oil may optionally be generated in the pyrolysis step, the present invention aims also at reducing as much as possible the generation of this first liquid oil, and instead keeping everything or as much as possible in the gas phase until after the HDO reactor of step i). Accordingly, in an embodiment there is no generation of a first liquid oil stream in step i), as for instance described farther below in connection with FIGS. 1 and 2. The first liquid oil stream is more reactive and thereby unstable than the second liquid oil stream. The first liquid oil stream presents therefore more challenges in downstream units, by way of i.a. its exothermicity and tendency to polymerize and plug the units.

For the purposes of the present invention, the pyrolysis step is preferably fast pyrolysis, also referred in the art as flash pyrolysis. Fast pyrolysis means the thermal decomposition of a solid renewable feedstock in the absence of oxygen, at temperatures in the range 350-650° C. e.g. about 500° C. and reaction times of 10 seconds or less, e.g. below 10 seconds, such as 5 seconds or less, e.g. about 2 seconds; i.e. the vapor residence time is 10 seconds or below, such as 2 seconds or less e.g. about 2 seconds. Traditionally, fast pyrolysis may for instance also be conducted by autothermal operation e.g. in a fluidized bed reactor. The latter is also referred as autothermal pyrolysis and is characterized by employing air, optionally with an inert gas or recycle gas, as the fluidizing gas, or by using a mixture of air and inert gas or recycle gas. Thereby, the partial oxidation of pyrolysis compounds being produced in the pyrolysis reactor (autothermal reactor) provides the energy for pyrolysis while at the same time improving heat transfer. For details about autothermal pyrolysis, reference is given to e.g. “Heterodoxy in Fast Pyrolysis of Biomass” by Robert Brown:
https://dx.doi.org/10.1021/acs.energyfuels.0c03512

Yet, in an embodiment of the present application, the use of autothermal pyrolysis. i.e. autothermal operation, as a particular embodiment for conducting fast pyrolysis, is omitted, i.e. the pyrolysis step is not conducted by autothermal pyrolysis.

There are several types of fast pyrolysis where a catalyst is used. Sometimes an acid catalyst, such as a zeolite catalyst, is used in the pyrolysis unit (pyrolysis reactor) to upgrade the pyrolysis vapors; this technology is called catalytic fast pyrolysis (CFP) and can both be operated in an in-situ mode (the catalyst is located inside the pyrolysis unit), and an ex-situ mode (the catalyst is placed in a separate reactor; i.e. the pyrolysis gas is sent to a deoxygenation (DO) reactor for catalytically deoxygenating it prior to condensation of a pyrolysis oil, as described farther above). More specifically, in in-situ catalytic fast pyrolysis the catalyst is located inside the pyrolysis unit and the deoxygenation (through e.g. decarbonylation, decarboxylation by an acid-based catalyst such as a zeolite catalyst) takes place inside the pyrolysis reactor immediately after the pyrolysis vapours are formed. Suitable catalysts for CFP include alumina and all the types of zeolite catalysts that are normally used for hydrocracking (HCR) and cracking in refinery processes, such as HZSM-5. A more extensive list of catalytic material for HCR is provided farther below in the present application.

Similarly, in in-situ HDO (also called reactive catalytic fast pyrolysis, RCFP), a hydrotreating (HDO) catalyst is located in the pyrolysis unit, and the pyrolysis vapors are thereby hydrodeoxygenated immediately in the pyrolysis reactor after they are formed. Suitably catalysts for HDO are metal-based catalysts, including reduced Ni, Mo, Co, Pt, Pd, Re, Ru, Fe, such as CoMo or NiMo catalysits, suitably also in sulfide form: CoMoS, NiS, NiMoS, NiWS, RuS. The catalyst supports may be the same in conventional HDO in refinery processes, typically a refractory support such as alumina, silica or titania, or combinations thereof. Farther below in the present application, HDO conditions are also recited.

In ex-situ deoxygenation (DO), the vapors are deoxygenated in a separate DO reactor located after the pyrolysis unit. Thus, in ex-situ catalytic fast pyrolysis, the vapors are deoxygenated using an acid catalyst, such as a zeolite catalyst.

In ex-situ HDO, the pyrolysis vapors are hydrodeoxygenated in a separate HDO reactor located after the pyrolysis reactor using a hydrotreating catalyst, as for instance described in connection with FIGS. 1 and 2 farther below.

The use of a catalyst in the pyrolysis reactor conveys the advantage of lowering the activation energy for reactions thereby significantly reducing the required temperature for conducting the pyrolysis. In addition, increased selectivity towards desired pyrolysis oil compounds may be achieved.

It would be understood that where hydrogen is added to the catalytic fast pyrolysis, it is called reactive catalytic fast pyrolysis (RCFP). Further, if the catalytic fast pyrolysis is conducted at a high hydrogen pressure (˜>5 barg) it is often called catalytic hydropyrolysis (CHP). Hydropyrolysis (HP) means that hydrogen is added to the pyrolysis, yet at atmospheric pressure.

The pyrolysis step is suitably also a simple fast pyrolysis, which for the purposes of this application means fast pyrolysis being conducted without the presence of a catalyst and hydrogen in the pyrolysis unit, i.e. the fast pyrolysis is not any of: catalytic fast pyrolysis (CFP), hydropyrolysis (HP), reactive catalytic fast pyrolysis (RCFP) or catalytic fast hydropyrolysis (CHP). The pyrolysis unit may not include a HDO reactor downstream. This enables a much simpler and inexpensive process.

The table below summarizes the different options for fast pyrolysis apart from autothermal pyrolysis:

| | Hydrogen | Catalyst | HDO within | DO within |
|---|---|---|---|---|
| Fast | added to | within | or outside | or outside |
| pyrolysis | pyrolysis | pyrolysis | pyrolysis | pyrolysis |
| type | unit | unit | unit | unit |
| Simple fast | no | no | outside | no |
| pyrolysis | | | (ex-situ | |
| | | | HDO) | |
| Simple fast | no | no | no | no |
| pyrolysis | | | | |
| in-situ CFP | no | yes (acid | no | Inside |
| | | catalyst) | | |
| ex-situ CFP | no | no | no | outside |
| | | | | (ex-situ |
| | | | | DO) |
| RCFP (in- | yes | yes (HDO | Inside | no |
| situ HDO) | | catalyst) | | |
| HP | yes | yes/no | outside | outside |
| CHP | yes, at | As for | As for | As for |
| | high H₂ | RCFP, | RCFP, | RCFP, |
| | pressure | HP | HP | HP |
| | (>5 barg) | | | |

Accordingly, in an embodiment the pyrolysis step is fast pyrolysis, in which the vapor residence time is 10 seconds or less, e.g. below 10 seconds, such as 5 seconds or less, e.g. about 2 seconds, or 1 second, or in the range 1-5 seconds, and which is selected from: simple fast pyrolysis; in-situ catalytic fast pyrolysis (in-situ CFP); ex-situ catalytic fast pyrolysis (ex-situ CFP); reactive catalytic fast pyrolysis (RCFP); hydropyrolysis (HP);
catalytic fast hydropyrolysis (CHP).

In another embodiment, the pyrolysis step is intermediate pyrolysis, in which the vapor residence time is in the range of 10 seconds-5 minutes, such as 11 seconds-3 minutes. As for fast pyrolysis, the temperature is also in the range 350-650° C. e.g. about 500° C. Often this pyrolysis is conducted in pyrolysis reactors handling different types of waste, where the vapor is burned after the pyrolysis reactor. Typical reactors are: Herreshoff furnace, rotary drums, amaron, CHOREN paddle pyrolysis kiln, auger reactor, and vacuum pyrolysis reactor.

In another embodiment, the pyrolysis step is slow pyrolysis, in which the solid residence time is in the range of 5 minutes-2 hours, such as 10 min-1 hour. The temperature is suitably about 300° C. This pyrolysis gives a high char yield and the char can be used as a fertilizer or as char coal; the pyrolysis still produces some gas and biocrude and if the carbon is used a fertilizer the final bio-oil can have a GHG above 100%, thus being carbon negative. Typical reactors are auger reactor—yet with a different residence time than for intermerdiate pyrolysis —, fixed bed reactor, kiln, lambiotte SIFIC/CISR retort, Lurgi process, wagon reactor, and carbo twin resort.

In an embodiment, said first off-gas stream comprises CO, CO₂ and light hydrocarbons such as C1-C4, and optionally also H₂S.

In an embodiment, the thermal decomposition step is a hydrothermal liquefaction step. Hydrothermal liquefaction means the thermochemical conversion of biomass into liquid fuels by processing in a hot, pressurized water environment for sufficient time to break down the solid biopolymeric structure to mainly liquid components. Typical hydrothermal processing conditions are temperatures in the range of 250-375° C. and operating pressures in the range of 40-220 bar. This technology offers the advantage of operation of a lower temperature, higher energy efficiency and lower tar yield compared to pyrolysis, e.g. fast pyrolysis. For details on hydrothermal liquefaction of biomass, reference is given to e.g. Golakota et al., “A review of hydrothermal liquefaction of biomass”, Renewable and Sustainable Energy Reviews, vol. 81, Part 1, Jan. 2018, p. 1378-1392.

In an embodiment, the thermal decomposition further comprises a preliminary step of passing said solid renewable feedstock through a solid renewable feedstock preparation section comprising for instance drying for removing water and/or comminution for reduction of particle size. Any water/moisture in the solid renewable feedstock which vaporizes in for instance the pyrolysis section condenses in the pyrolysis oil stream and is thereby carried out in the process, which may be undesirable. Furthermore, the heat used for the vaporization of water withdraws heat which otherwise is necessary for the pyrolysis. By removing water and also providing a smaller particle size in the solid renewable feedstock the thermal efficiency of the pyrolysis step is increased.

The preliminary step may also comprise conducting an acid wash for removing metals. This is particularly relevant for pyrolysis processes where the catalyst is located in the pyrolysis reactor. The removal of metals from the solid renewable feedstock increases the catalyst lifetime.

In an embodiment, the solid renewable feedstock is a lignocellulosic biomass including wood products, forestry waste, and agricultural residue. In another embodiment the solid renewable feedstock is municipal waste, in particular the organic portion thereof.

For the purposes of the present application, the term “municipal waste” is interchangeable with the term “municipal solid waste” and means a feedstock containing materials of items discarded by the public, such as mixed municipal waste given the waste code 200301 in the European Waste Catalog.

In a particular embodiment, the lignocellulosic biomass is forestry waste and/or agricultural residue and comprises biomass originating from plants including grass such as nature grass (grass originating from natural landscape), wheat e.g. wheat straw, oats, rye, reed grass, bamboo, sugar cane or sugar cane derivatives such as bagasse, maize and other cereals.

Any combinations of the above are also envisaged.

As used herein, the term “lignocellulosic biomass” means a biomass containing, cellulose, hemicellulose and optionally also lignin. The lignin or a significant portion thereof may have been removed, for instance by a prior bleaching step.

In an embodiment, electrolysis in step iv) is conducted in a solid oxide electrolysis cell (SOEC) unit. In another embodiment, the electrolysis is conducted in an alkaline and/or PEM electrolysis unit. Liquid water is used in the latter, while steam is used in the SOEC unit. As steam is already available in the process, e.g. it is generated in the process, the use of SOEC is advantageous. Steam may also be imported from outside plant battery limits, if required. The power for the water (steam) electrolysis is suitably derived from wind, hydropower and/or solar energy, thus enabling the production of hydrogen from renewable sources.

In an embodiment, the steam reforming step in step iii-2) is conducted in an electrically heated reformer (e-reformer), i.e. the steam reforming unit is an e-reformer. The term “e-reformer” and “e-SMR” for electrically heated steam methane reformer, are used interchangeably. The e-reformer is suitably also powered by electricity derived from renewable resources such as wind, hydropower and/or solar energy. For a description of e-SMR which is a recent technology, reference is given to in particular applicant's WO 2019/228797 A1.

In an embodiment, between step i) and ii) or between step ii) and iii), said first off-gas stream or said upgraded first off-gas stream or said further upgraded first off-gas stream, passes to a separation unit for sulfur removal, the separation unit preferably being at least one of an amine absorption unit, a caustic scrubber, and a sulfur absorbent unit. Sulfur, in particular H₂S is detrimental for catalysts used in downstream operations, and thus is suitably removed.

In an embodiment, the process further comprises:
conducting in step iii-2):
   an optional water gas shift conversion step,
   a CO₂ removal step after the steam reforming step for producing said methanol synthesis gas;
or
conducting between step ii) and iii) a water gas shift conversion step and a CO₂ removal step.

As is well known in the art, the water gas shift conversion serves to enrich the synthesis gas from the steam reforming into hydrogen according to the catalytic reaction CO+H₂O=H₂+CO₂. The CO₂ is suitably removed by any conventional method, such as by membrane or cryogenic separation or pressure swing adsorption (CO₂—PSA) or amine absorption unit or methanol wash (Rectisol/Selexol).

The present invention converts pyrolysis gas into methane or methanol where the required hydrogen is produced by electrolysis, in particular in a SOEC unit. In cases where the thermal decomposition step is a pyrolysis step comprising the use of a pyrolysis unit requiring hydrogen, such as in catalytic hydropyrolysis (CHP), the SOEC also is capable of producing the hydrogen needed in the pyrolysis. Steam generated during the production of methanol or methane is used in the SOEC, while hydrogen produced in the SOEC is also used in the thermal decomposition step, for instance in the pyrolysis unit or in the HDO, as well as the methanation or methanol synthesis, thereby enabling high integration of the different process steps.

By “integration” is meant that the different process steps and thereby associated units are in fluid communication with the electrolysis unit.

The present invention provides also a bridge between so-called power-to-X and bio-to-X technologies. The present invention enables the use of renewable electricity, e.g. from wind or solar, optionally thermonuclear energy, to power the electrolysis to produce hydrogen, which is then used to produce e.g. methane or methanol and thereby gasoline from an undesirable byproduct (first off-gas stream, i.e. the first pyrolysis off-gas) from the thermal decomposition of a solid renewable feedstock, thereby increasing the overall product value and carbon recovery as explained farther above. Moreover, the present invention enables the production of both e-methane or e-methanol, as well as biofuels i.e. fuels such as diesel and jet fuel, as described below, from a solid renewable feedstock, thereby meeting stricter demands according to e.g. the Renewable Energy Directive II (RED II) under the European Union.

Furthermore, where a pyrolysis oil (first liquid oil stream) is also generated in step i), the invention enables in a single process and plant, that the carbon in the solid renewable feedstock, including the carbon withdrawn as char in the pyrolysis step, is nearly 100% recovered as the valuable products methane e.g. as substitute natural gas (SNG) and methanol, optionally also as gasoline, as well as additional valuable hydrocarbons in the transportation fuel range such as diesel and jet fuel, as it will become apparent from one or more of the below embodiments. As methanol can be further converted to gasoline, the invention enables therefore the production of the full range of transportation fuels: diesel, jet fuel and gasoline, as well as maritime (marine) fuel, all derived from the same original solid renewable feedstock.

Accordingly, in an embodiment the process further comprises:
vi) conducting the second liquid oil stream of step i) and/or the optional first liquid oil stream of step i), optionally also at least a portion of the hydrogen stream from the electrolysis step (step iv), to a hydroprocessing step for producing a main hydroprocessed stream;
vii) conducting the main hydroprocessed stream to a separation step for producing:
an aqueous stream,
a hydrogen-rich stream,
a second off-gas stream comprising hydrocarbons,
and a hydrocarbon product, boiling at above 50° C.;
viii) conducting the second off-gas stream from step vii) to said olefin removal step i.e. step ii), or to said step iii), and/or to a hydrogen producing unit (HPU) for producing a make-up hydrogen stream;
the process further comprising:
conducting the hydrogen-rich stream from step vii), or a portion thereof, to the hydroprocessing step, without subjecting said hydrogen-rich stream, or the portion thereof, to a separation step for removing H₂S and/or CO₂ optionally also for removing NH₃ and/or CO.

Hence, the second liquid oil stream, or the first liquid oil stream, or a combination thereof e.g. as co-feed stream, is treated in a refinery section (refinery process/plant) comprising a hydroprocessing section for producing the main hydroprocessed stream in accordance with step vi), and a separation section downstream for producing i.a. the hydrogen-rich stream and the hydrocarbon products such as diesel and jet fuel, in accordance with step vii).

Thereby, high integration of the process/plant for producing methane or methanol from a pyrolysis gas and a pyrolysis solid carbon stream, with the refinery process/plant is provided.

While the first liquid oil stream may contain a significant amount of chemically bound oxygen, for instance up to 30 or 40 wt %, the second liquid oil stream is derived from a stream (first off-gas stream from the thermal decomposition step, e.g. pyrolysis) which has been subjected to e.g. HDO already. The co-feeding of the second liquid oil stream or a portion thereof with the first liquid oil stream or a portion thereof enables therefore the provision of a heat sinking effect thereby reducing the exothermicity in the hydroprocessing step treating the first liquid oil stream, in particular a stabilization and HDO step therein, as the first liquid oil stream (from e.g. the pyrolysis unit) is much richer in oxygen and thus more reactive than the second liquid oil stream.

From the separation step, i.e. from a separation section, a second off-gas stream is also produced, which is then conducted to the olefin removal step and thereby further converted to methane or methanol. The second off-gas is e.g. conducted to the HPU to generate in the latter make-up hydrogen that can be used in the process, for instance as make-up hydrogen in the hydroprocessing section. It would be understood that a portion of the second off-gas stream may be conducted to the olefin removal step and/or to the HPU. Hydrogen produced in the electrolysis step is suitably also used as make-up hydrogen in the hydroprocessing section. Further integration is thereby achieved.

Furthermore, the incorporation of the HPU provides for increased flexibility in the process: renewables sources such as wind and solar are intermittent; thus, when there is plenty of sun or wind for producing electricity, the electrolysis unit provides for most or all of the hydrogen required in the process, while when there is no sun or wind for producing electricity in sufficient amounts, the HPU is used for producing hydrogen i.e. make-up hydrogen. As for the instance of hydrogen produced from the electrolysis step, at least a portion of the make-up hydrogen is suitably conducted to any the: thermal decomposition step including HDO-step, olefin hydrogenation step, methanation step, methanol synthesis step, or combinations thereof.

As used herein, the term “section”, for instance “hydroprocessing section”, means a physical section comprising a unit or combination of units for conducting one or more steps and/or sub-steps for producing said main hydroprocessed stream. It would be understood that this corresponds to the hydroprocessing step.

As used herein, the term “hydrogen producing unit” means a hydrogen producing section. Hence, the hydrogen producing unit (HPU) means also a physical section comprising a unit or combination of units for conducting one or more steps and/or sub-steps during the production of the make-up hydrogen stream.

By the present invention, the second off-gas generated in the separation step vii) is suitably converted to hydrogen in the HPU, or into methanol or methane, thereby providing yet further integration in the process/plant.

In an embodiment, said hydrocarbon product, boiling at above 50° C., is a hydrocarbon product boiling at least in one of: the diesel fuel boiling range, jet fuel boiling range, and naphtha boiling range.

In an embodiment, the hydrocarbon product is a maritime (marine) fuel.

In an embodiment, said second off-gas stream comprises light hydrocarbons in the form of C1-C4 hydrocarbons, H₂, CO, CO₂, and optionally also H₂S.

In an embodiment, said hydrogen-rich stream (in step vii) comprises 50% vol. H₂ or more, light hydrocarbons such as C1-C4 hydrocarbons, optionally also H₂S and NH₃, CO and CO₂.

This hydrogen-rich stream which is produced in the separation step vii) is suitably used in the prior hydroprocessing step, i.e. recycle to the hydroprocessing step. Accordingly, by the present application, the process further comprises: conducting the hydrogen-rich stream from step vii), or a portion thereof, to the hydroprocessing step, without subjecting said hydrogen-rich stream to a separation step for removing H₂S and/or CO₂ optionally also for removing NH₃ and/or CO.

In an embodiment, prior to conducting step viii), said second off-gas stream from step vii) passes to a separation unit, the separation unit preferably being at least one of an amine absorption unit, a caustic scrubber, and a sulfur absorbent unit, for removing H₂S. The resulting gas stream entering the HPU contains therefore light hydrocarbons such as C1-C4 hydrocarbons, H₂, NH₃, CO and CO₂, yet no H₂S or only minor amounts of H₂S. The second off-gas stream and the gas stream derived thereof after passing through the separation unit, contains hydrogen not consumed from the hydrotreating unit(s) of the hydroprocessing stage as soluble hydrogen in hydrocarbon phase and is suitably used as part of the feed in the hydrogen producing unit, which is described farther below.

Suitably, the separation unit for treating the second off-gas stream from step vii) is the same separation unit for sulfur removal used for said upgraded first of-gas stream or said further upgraded first off-gas stream. That is, the separation unit is preferably the at least one of an amine absorption unit, a caustic scrubber, and a sulfur absorbent unit, for removing H₂S, adapted between step i) and ii) (i.e. prior to olefin removal) or between step ii) and iii) (i.e. prior to methanation or steam reforming). Higher integration and flexibility in the process is thereby achieved.

Traditionally in refineries, the hydrogen-rich stream generated in the separation step (step vii) is recycled to the hydroprocessing step and for this purpose it is first subjected to a separation for removing H₂S and/or CO₂, optionally also for removing NH₃ and/or CO, prior to being passed to the hydroprocessing step.

In contrast thereof, as recited above, the hydrogen-rich stream from step vii) is not subjected to a separation step for removing H₂S and/or CO₂, optionally also for removing NH₃ and/or CO, when conducting said hydrogen-rich stream, or a portion thereof, to the hydroprocessing step.

The hydrogen-rich stream produced in the separation step vii) is significantly larger i.e. significantly larger flow rate, than the second off-gas stream produced in this step, thus the provision of a separation stage such as an amine scrubber in the hydrogen-rich stream for removing H₂S and/or CO₂, often for removing H₂S and CO₂, is by the present invention obviated, without incurring any penalty in the process, for instance by using a nickel-molybdenum catalyst for hydrodeoxygenation in the hydroprocessing step as in applicant's co-pending patent application EP 20162755.1. Furthermore, renewable feeds such as vegetable oil, animal fat etc., which may be co-fed, often lack enough sulfur compared with conventional fossil feed. As a result, an external sulfur agent such as dimethyl disulfide (DMDS) or other sulfur agent has normally been introduced with conventional fossil feed to provide the minimum required H₂S amount in the hydrogen rich gas to hydrodeoxygenation of said hydroprocessing step to keep the hydrotreating e.g. hydrodeoxygenation catalyst therein in sulfided form. So, use of high pressure amine absorber will remove the H₂S from hydrogen-rich gas prior to sending back to the hydroprocessing step. This results in more addition of external sulfur agent and added cost.

A separation unit such as an amine scrubber is thus suitably provided in the much smaller second off-gas stream and targeted for H₂S removal, thereby simplifying the process and reducing capital and operating expenses, as well as reducing energy consumption, by virtue of using a smaller separation stage in a smaller stream, i.e. the second off-gas stream, as described in applicant's co-pending patent application PCT/EP2021/056085. More specifically, there is less amine, e.g. lean amine, requirement thus resulting in a smaller amine regeneration unit and less amount of steam required to regenerate the amine. In addition, the amine scrubber in the second off-gas stream is suitably a low pressure amine absorption system, which conveys much lower capital and operating expenses compared to high pressure amine absorption systems which are normally used when cleaning the hydrogen-rich gas stream prior to passing it to said hydrodeoxygenation in the hydroprocessing step.

Furthermore, while it is also possible to remove the CO₂ in the second off-gas stream, suitably the CO₂ is not removed for avoiding the emission of CO₂ to the atmosphere, since the hydrogen producing unit, e.g. a pre-reforming unit therein, can operate with the second recycle also containing CO₂. A lower carbon footprint is thereby obtained.

Where necessary, an amine which is more selective towards H₂S removal can be selected, with CO₂ removal being incidental. The removal of H₂S from the second off-gas stream minimizes the need for sulfur adsorbent in the hydrogen producing unit, in particular a cleaning unit therein.

In an embodiment, the HPU comprises subjecting the second off-gas stream to: cleaning in a cleaning unit, said cleaning unit preferably being a sulfur-chlorine-metal absorption or catalytic unit; optionally pre-reforming in a pre-reforming unit; catalytic steam methane reforming in a steam reforming unit, suitably an e-reformer; water gas shift conversion in a water gas shift unit; optional carbon dioxide removal in a CO₂-separator unit, optional hydrogen purification in a hydrogen purification unit.

Hence, the second off-gas stream is used as hydrocarbon feed to the HPU.

In an embodiment, the process further comprises:
ix) conducting a portion of the first off-gas stream of step i) to the HPU;
and/or
x) producing in step iii-2) a methanol synthesis purge gas, suitably comprising CH₄, H₂, CO, CO₂, CH₃OH and inerts such as N₂, and conducting at least a portion of the methanol synthesis purge gas to the HPU;
and/or
xi) further producing in the separation step vii) an LPG stream and feeding the LPG stream to the HPU.

The use of a portion of the first off-gas from step i), e.g. a pyrolysis off-gas, in accordance with step ix) enables relieving the hydrocarbon feed gas requirement in the HPU, as described in applicant's co-pending patent application EP 21152112.5. The hydrocarbon feed gas for a HPU is normally natural gas, yet its use should be minimized for not least environmental reasons. The present invention enables use of naphtha producing in step vii) as one of the hydrocarbon products, instead of using it as hydrocarbon feed gas (make-up gas) in the HPU for replacing at least part of the natural gas required in the HPU. Thus, the use of part of the pyrolysis gas replaces the need of using valuable naphtha produced in the process, so that the latter instead of being “sacrificed” by using it as hydrocarbon feed gas in the HPU, can be further upgraded e.g. via a subsequent aromatization step, to gasoline.

When conducting the steam methane reforming by for instance SMR or HTCR (see description farther below) there is a hydrocarbon feed gas and a fuel gas, whereby the latter is suitably burned to give the necessary heat input to the SMR or HTCR process.

The methanol synthesis purge gas is by the present invention also advantageously used as fuel gas in order to avoid recycle of inert gases such as N₂.

The separation in step vii) also produces an LPG stream and the process further comprises feeding the LPG stream to the hydrogen producing unit. Thereby, there is less need for natural gas as external source for providing hydrocarbon feed gas to the HPU.

As used herein, “LPG” means liquid petroleum gas (also referred as liquified petroleum gas), which is a gas mixture mainly comprising propane and butane, i.e. C3-C4; LPG may also comprise i-C4 and unsaturated C3-C4 such as C4-olefins.

In an embodiment, natural gas is also used as part of the hydrocarbon feed i.e. make-up gas, to the HPU to assist in the hydrogen production.

In a particular embodiment, the second off-gas stream is fed to the cleaning unit, i.e. to the cleaning unit of the HPU. This is an efficient way of utilizing available hydrogen in the process, as the sulfur-chlorine-metal absorption or catalytic unit normally requires addition of hydrogen.

The hydrogen purification unit may be a H₂-membrane separation unit, or a Pressure Swing Adsorption unit (PSA-unit). The PSA generates normally a PSA off-gas which is suitably used in the process, for instance as fuel gas for providing for the above-mentioned indirect heating in step i-2).

In an embodiment, the steam reforming unit is: a convection reformer, preferably comprising one or more bayonet reforming tubes such as an HTCR reformer i.e.

Topsoe bayonet reformer, where the heat for reforming is transferred by convection along with radiation; a tubular reformer i.e. conventional steam methane reformer (SMR), where the heat for reforming is transferred chiefly by radiation in a radiant furnace; autothermal reformer (ATR), where partial oxidation of the hydrocarbon feed with oxygen and steam followed by catalytic reforming; electrically heated steam methane reformer (e-SMR) i.e. e-reformer, where electrical resistance is used for generating the heat for catalytic reforming; or combinations thereof. In particular, when using an e-reformer, electricity from green resources may be utilized, such as from electricity produced by wind power, hydropower, and solar sources, thereby further minimizing the carbon dioxide footprint.

For more information on these reformers, details are herein provided by direct reference to Applicant's patents and/or literature. For instance, for tubular and autothermal reforming an overview is presented in “Tubular reforming and autothermal reforming of natural gas—an overview of available processes”, lb Dybkjmr, Fuel Processing Technology 42 (1995) 85-107; and EP 0535505 for a description of HTCR. For a description of ATR and/or SMR for large scale hydrogen production, see e.g. the article “Large-scale Hydrogen Production”, Jens R. Rostrup-Nielsen and Thomas Rostrup-Nielsen”: https://www.topsoe. com/sites/default/files/topsoe_large_scale_hydrogen_produc.pdf Alternatively, for a description of ATR and/or SMR for large scale hydrogen production, see e.g. the article “Large-scale Hydrogen Production”, Jens R. Rostrup-Nielsen and Thomas Rostrup-Nielsen”, CATTECH 6, 150-159 (2002). For a description of e-SMR (e-reformer) which is a more recent technology, reference is given to in particular applicant's WO 2019/228797 A1, as already mentioned farther above.

In an embodiment, the catalyst in the steam reforming unit is a reforming catalyst, e.g. a nickel-based catalyst. In an embodiment, the catalyst in the water gas shift reaction is any catalyst active for water gas shift reactions. The said two catalysts can be identical or different. Examples of reforming catalysts are Ni/MgAl₂O₄, Ni/Al₂O₃, Ni/CaAl₂O₄, Ru/MgAl₂O₄, Rh/MgAl₂O₄, Ir/MgAl₂O₄, Mo₂C, Wo₂C, CeO₂, Ni/ZrO₂, Ni/MgAl₂O₃, Ni/CaAl₂O₃, Ru/MgAl₂O₃, or Rh/MgAl₂O₃, a noble metal on an Al₂O₃ carrier, but other catalysts suitable for reforming are also conceivable. The catalytically active material may be Ni, Ru, Rh, Ir, or a combination thereof, while the ceramic coating may be Al₂O₃, ZrO₂, MgAl₂O₃, CaAl₂O₃, or a combination therefore and potentially mixed with oxides of Y, Ti, La, or Ce. The maximum temperature of the reactor may be between 850-1300° C. The pressure of the feed gas may be 15-180 bar, preferably about 25 bar.

Steam reforming catalyst is also denoted steam methane reforming catalyst or methane reforming catalyst.

In an embodiment, in step vi the hydroprocessing step comprises:
vi-1) conducting said second liquid oil stream and/or said first liquid oil stream, suitably a combination thereof, to a first catalytic hydrotreating unit under the addition of hydrogen for producing a first hydrotreated stream, e.g. comprising C1-C65 hydrocarbons;
vi-2) conducting the first hydrotreated stream to a dewaxing step comprising a second catalytic hydrotreating unit under the addition of hydrogen for producing said main hydrotreated stream;
wherein prior to conducting step vi-1), suitably prior to conducting to a hydrodeoxygenation (HDO) unit, said second liquid oil stream and/or said first liquid oil stream is conducted to a catalytic unit for liquid oil stabilization under the addition of hydrogen.

Optionally, the hydroprocessing step comprises using one or more additional catalytic hydrotreating units under the addition of hydrogen, such as third catalytic hydrotreating unit or a cracking section. For instance, it would be understood that when a hydrocarbon product boiling in the jet fuel range is desired, a hydrocracking unit is suitably used, for instance prior to passing the thus resulting first hydrotreated stream to the dewaxing.

In an embodiment, the first catalytic hydrotreating unit is hydrodeoxygenation (HDO), the second catalytic hydrotreating is hydrodewaxing/hydroisomerization (HDW/HDI), and an additional catalytic hydrotreating such as a third catalytic hydrotreating is hydrocracking (HCR).

It would be understood, that that term “dewaxing” is used interchangeably with the term “hydrodewaxing/hydrosiomerization (HDW/HDI)”.

Normally the pyrolysis oil contains a high amount of oxygen compound and unsaturated hydrocarbon. During the hydrotreating of this feed, the oxygen is mainly removed as H₂O, which gives a fuel consisting of mainly naphthenes and aromatics. This is called the hydrodeoxygenation (HDO) pathway. Oxygen can also be removed by the decarboxylation pathway, which generates CO₂ instead of H₂O: HDO pathway: RCH₂COOH+3H₂↔RCH₂CH₃+2H₂O Decarboxylation pathway: RCH₂COOH↔RCH₃+CO₂

Further, while decarbonylation normally does not occur in HDO of triglycerides in typical renewable feeds, it can occur during HDO of pyrolysis oil: Decarbonylation pathway: RCH₂COH+H₂<->RCH₃+CO The material catalytically active in HDO (as used herein, interchangeable with the term hydrotreating, HDT), typically comprises an active metal (sulfided base metals such as nickel, cobalt, tungsten and/or molybdenum, but possibly also either elemental noble metals such as platinum and/or palladium) and a refractory support (such as alumina, silica or titania, or combinations thereof).

HDT conditions involve a temperature in the interval 250-400° C., a pressure in the interval 30-150 bar, and a liquid hourly space velocity (LHSV) in the interval 0.1-2, optionally together with intermediate cooling by quenching with cold hydrogen, feed or product

The material catalytically active in hydrodewaxing/hydroisomerization HDW/HDI typically comprises an active metal (either elemental noble metals such as platinum and/or palladium or sulfided base metals such as nickel, cobalt, tungsten and/or molybdenum), an acidic support (typically a molecular sieve showing high shape selectivity, and having a topology such as MOR, FER, MRE, MWW, AEL, TON and MTT) and a refractory support (such as alumina, silica or titania, or combinations thereof).

HDW/HDI conditions involve a temperature in the interval 250-400° C., a pressure in the interval 20-100 bar, and a liquid hourly space velocity (LHSV) in the interval 0.5-8.

The material catalytically active in hydrocracking HCR is of similar nature to the material catalytically active in isomerization, and it typically comprises an active metal (either elemental noble metals such as platinum and/or palladium or sulfided base metals such as nickel, cobalt, tungsten and/or molybdenum), an acidic support (typically a molecular sieve showing high cracking activity, and having a topology such as MFI, BEA and FAU) and a refractory support (such as alumina, silica or titania, or combinations thereof). The difference to material catalytically active isomerization is typically the nature of the acidic support, which may be of a different structure (even amorphous silica-alumina) or have a different acidity e.g. due to silica:alumina ratio.

Hydrocracking HCR conditions involve a temperature in the interval 250-400° C., a pressure in the interval 30-150 bar, and a liquid hourly space velocity (LHSV) in the interval 0.5-8, optionally together with intermediate cooling by quenching with cold hydrogen, feed or product

Other types of hydrotreating are also envisaged, for instance hydrodearomatization (HDA). The material catalytically active in hydrodearomatization typically comprises an active metal (typically elemental noble metals such as platinum and/or palladium but possibly also sulfided base metals such as nickel, cobalt, tungsten and/or molybdenum) and a refractory support (such as amorphous silica-alumina, alumina, silica or titania, or combinations thereof).

Hydrodearomatization conditions involve a temperature in the interval 200-350° C., a pressure in the interval 20-100 bar, and a liquid hourly space velocity (LHSV) in the interval 0.5-8.

By the term “stabilization” is meant converting carbonyl groups present in compounds of the liquid oil, such as aldehydes, ketones and acids, into alcohols. Other molecules such as sugars and furans are also converted in the stabilization step. For instance, this stabilization step can be conducted by means of NiMo based catalysts, as disclosed in Shumeico et al. “Efficient one-stage bio-oil upgrading over sulfide catalysts”, ACS Sustainable Chem. Eng. 2020, 8, 15149-15167. As used herein, this stabilization step is included in the hydroprocessing step vi). In a particular embodiment, the stabilization is conducted according to the method disclosed in Applicant's co-pending European patent application 21152117.4

In an embodiment, the process comprises: conducting at least a portion of the hydrogen stream from the electrolysis step (step iv) to the hydroprocessing step vi), suitably to said catalytic unit for liquid oil stabilization. The hydrogen from the electrolysis is suitably also added to the one or more catalytic units of the hydroprocessing step, such as the HDO unit. Thereby further integration in the process is achieved. Furthermore, there is greater flexibility in the supply of needed make-up hydrogen for the process without resorting to external sources.

In an embodiment, between step vi-1) and vi-2) the process further comprises conducting the first hydrotreated stream to a separator, such as a high-pressure or low-pressure separator, for removing H₂S, NH₃, and H₂O, thereby producing said first hydrotreated stream, and optionally also producing a vapor stream, and a recycle oil stream.

The first hydrotreated stream from the first catalytic hydrotreating unit normally contains impurities, in particular H₂S, NH₃, CO and CO₂ which may be detrimental for the catalyst used in the subsequent dewaxing section. When the process is operated in so-called sour mode, the catalyst of the dewaxing/hydroisomerization step is a base-metal catalyst, which is resistant to impurities, thereby avoiding the need of using a separator. When operating in the so-called sweet mode, the catalyst of the dewaxing section is a noble-metal catalyst, which is sensitive to the impurities, thereby requiring the need of using the separator.

In an embodiment, the separation step vii) comprises:
vii-1) conducting the main hydrotreated stream to a separator, preferably a cold separator, for producing said aqueous stream, said hydrogen-rich stream, and a heavy hydrocarbon stream, e.g. a hydrocarbon stream comprising C5-C30 hydrocarbons, H₂, CO and CO₂;
vii-2) conducting the heavy hydrocarbon stream to a fractionation step for producing said second off-gas stream and said hydrocarbon product, e.g. a hydrocarbon product boiling at least in one of: the diesel fuel boiling range, jet fuel boiling range, and naphtha boiling range, optionally also a maritime (marine) fuel.

The marine fuel is suitably separated as a heavy fuel oil.

Thereby, apart from methane (SNG) or methanol, further valuable products which are in the transportation fuel range are further produced from the same solid renewable feedstock, while at the same time providing a high integration in the overall process/plant. Naphtha is suitably further converted to gasoline, as already recited farther above.

It would be understood that by “overall process/plant” is meant the process and plant adapting steps i) to v) for producing methane or methanol, along with refinery steps vi)-viii), including any other intermediate and/or subsequent steps, in accordance with any of the above or below embodiments, i.e. in accordance with the appended claims.

In an embodiment, step vi) (the hydroprocessing step), further comprises co-feeding to the hydroprocessing step:
a first generation renewable feedstock selected from the group of renewable feeds which is already liquid, such as algae oil, virgin oil, rapeseed oil and soybean oil; and/or
a second generation renewable feedstock selected from the group of waste oil and fats: such as cooking oil, animal fats and crude tall oil (CTO); and/or
a feedstock comprising any of refused derived fuel (RDF) including municipal solid waste, plastic derived oil, and tire pyrolysis oil; and/or
a feedstock of fossil fuel origin, such as diesel, kerosene, naphtha, and vacuum gas oil (VGO), and/or recycling a hydrocarbon product e.g. recycle oil.

The co-feed acts as a hydrocarbon diluent, thereby further enabling the absorption of heat from the exothermal reactions in the catalytic hydrotreating unit(s), e.g. the HDO unit in the hydroprocessing step. The inventive process can thereby also be applied together with the hydroprocessing (hydrotreatment) of first and second-generation renewables, and optionally also third generation renewables.

In a second aspect, the invention encompasses also a plant for carrying out the process according to any of the embodiments according to the first aspect of the invention.

There is therefore also provided a plant for producing methanol and optionally a hydrocarbon product, the plant comprising:
a pyrolysis unit or hydrothermal liquefaction unit, arranged to receive a solid renewable feedstock and provide a first off-gas stream, a solid carbon stream (char stream), and optionally a first liquid oil stream;
a HDO/DO unit, i.e. hydrodeoxygenation or deoxygenation unit, arranged to receive, in the absence of steam, the first off-gas stream, and provide a HDO/DO treated stream; and a downstream separator arranged to receive the HDO/DO treated stream and provide a water stream, a second liquid oil stream, and an upgraded first off-gas stream;
an olefin removal reactor arranged to receive the upgraded first off-gas and provide a further upgraded first off-gas stream which is free of olefins;
a methanation reactor arranged to receive the further upgraded first off-gas stream and provide, under the generation of steam, a methane product stream;
or
a reforming unit arranged to receive the further upgraded first off-gas stream and provide a methanol synthesis gas, and a downstream methanol synthesis unit arranged to receive the methanol synthesis gas and to provide, under the generation of steam, a methanol product stream;
an electrolysis unit arranged to receive at least a portion of the steam generated in the methanation reactor or reforming unit or methanol synthesis unit, and provide an oxygen stream and hydrogen stream;
a conduit for supplying at least a portion of the hydrogen stream from the electrolysis unit to any of the: pyrolysis unit or hydrothermal liquefaction unit, the HDO unit, olefin removal reactor, methanation reactor, methanol synthesis units, or combinations thereof.

Thereby, all carbon in the solid renewable stock, apart from the solid carbon stream (char) withdrawn in step i) in connection with the thermal decomposition, is advantageously converted into the valuable products methane or methanol, as well as liquid oil from which hydrocarbon products may be generated

In an embodiment, the HDO/DO unit is arranged to provide an upgraded first off-gas stream which is less than 80 wt % of said solid renewable feedstock.

In an embodiment according to the second aspect of the invention, the plant further comprises producing a hydrocarbon product, and thus the plant further comprises:
a hydroprocessing section arranged to receive the second liquid oil stream and/or the optional first liquid oil stream and provide a main hydroprocessed stream;
a separation section arranged to receive the main hydroprocessed stream and provide: an aqueous stream, a hydrogen-rich stream, a second off-gas stream comprising hydrocarbons, and said hydrocarbon product, such as a hydrocarbon product boiling at above 50° C.;
a conduit for supplying the second off-gas stream from step vii) to said olefin removal reactor; and/or to a hydrogen producing unit (HPU) to provide a make-up hydrogen stream;
a conduit for conducting the hydrogen-rich stream or a portion thereof, to the hydroprocessing section, without providing a separation unit therein for removing H₂S and/or CO₂ optionally also for removing NH₃ and/or CO, from the hydrogen-rich stream or portion thereof.

Thereby, as in connection with the first aspect of the invention, the plant enables internally sourcing sulfur required in the hydrprocessing units of the hydroprocessing section, while at the same time avoiding the use of a dedicated separation unit such as an amine absorber on the hydrogen-rich stream being recycled to the hydroprocessing section.

Thus, as recited in connection with the first aspect of the invention, the hydrogen-rich stream produced is significantly larger i.e. significantly larger flow rate, than the second off-gas stream and which is produced in a fractionation section arranged further downstream in the separation section. Thus, the provision of a separation unit such as an amine scrubber in the hydrogen-rich stream for removing H₂S and/or CO₂, often for removing H₂S and CO₂, is by the present invention obviated, without incurring any penalty in the process, for instance by using a nickel-molybdenum catalyst for hydrodeoxygenation in the hydroprocessing step as in applicant's co-pending patent application EP 20162755.1. Furthermore, renewable feeds which may be co-fed, often lack enough sulfur compared with conventional fossil feed. As a result, an external sulfur agent such as dimethyl disulfide (DMDS) or other sulfur agent has normally been introduced with conventional fossil feed to provide the minimum required H₂S amount in the hydrogen rich gas to hydrodeoxygenation of said hydroprocessing step to keep the hydrotreating e.g. hydrodeoxygenation catalyst therein in sulfided form. So, use of high pressure amine absorber will remove the H₂S from hydrogen-rich gas prior to sending back to the hydroprocessing step. This results in more addition of external sulfur agent and added cost.

In an embodiment according to the second aspect of the invention, the hydroprocessing section comprises:
a catalytic unit for liquid oil stabilization under the addition of hydrogen, said catalytic unit being arranged to receive said second liquid oil stream and/or said first liquid oil stream, and provide a stabilized liquid oil;
a first catalytic hydrotreating unit arranged to receive the stabilized liquid oil and provide a first hydrotreated stream.

Hence, the second liquid oil stream, or the first liquid oil stream, or a combination thereof e.g. as co-feed stream, is treated in a refinery section (refinery process/plant) comprising the hydroprocessing section for producing the main hydroprocessed stream and a separation section downstream for producing i.a. the hydrogen-rich stream and the hydrocarbon products such as naphtha, jet fuel, diesel, or a marine fuel. Thereby, there is enabled a high integration of the process/plant for producing methane or methanol from a pyrolysis gas and a pyrolysis solid carbon stream, with the refinery process/plant is provided.

Further, while the first liquid oil stream may contain a significant amount of chemically bound oxygen, for instance up to 30 or 40 wt %, the second liquid oil stream is derived from a stream (first off-gas stream from the thermal decomposition step, e.g. pyrolysis) which has been subjected to e.g. HDO already. The co-feeding of the second liquid oil stream or a portion thereof with the first liquid oil stream or a portion thereof enables therefore the provision of a heat sinking effect thereby reducing the exothermicity in the hydroprocessing step treating the first liquid oil stream, in particular a stabilization and HDO step therein, as the first liquid oil stream (from e.g. the pyrolysis unit) is much richer in oxygen than the second liquid oil stream.

It would be understood that any of the embodiments of the first aspect of the invention and associated benefits may be used together with the second aspect of the invention, or vice versa.

### BRIEF DESCRIPTION OF THE DRAWINGS

FIG. 1 shows a schematic flow diagram of a process and plant for producing methane (Substitute Natural Gas, SNG) comprising pyrolysis, pyrolysis off-gas upgrading, methanation and electrolysis, in accordance with an embodiment of the invention.

FIG. 2 shows a schematic flow diagram of a process and plant for producing methanol comprising pyrolysis, pyrolysis off-gas upgrading, steam reforming and methanol synthesis, as well as electrolysis, in accordance with another embodiment of the invention.

FIG. 3 shows a schematic flow diagram of the overall process/plant, i.e. including a refinery section for conversion of the liquid oil streams produced in the process into hydrocarbon fuels such as diesel and jet fuel, in accordance with another embodiment of the invention.

### DETAILED DESCRIPTION

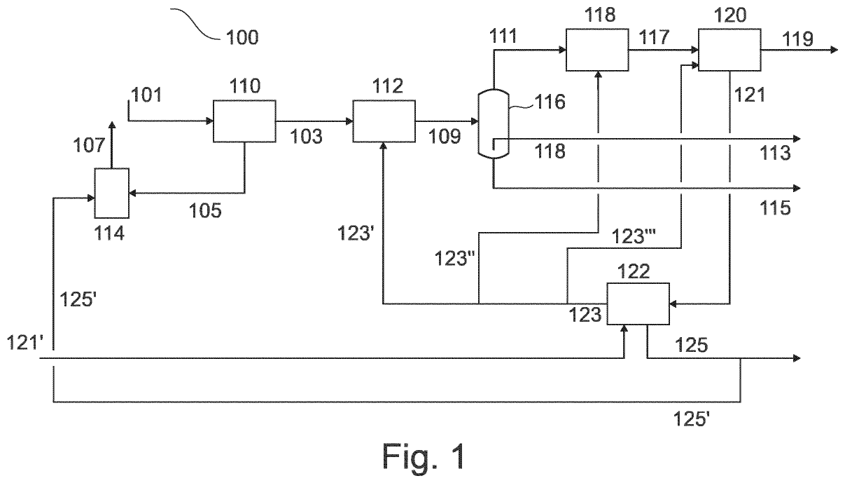

With reference to FIG. 1, a process/plant layout 100 for producing methane (SNG) is shown. A solid renewable feedstock 101 is conducted to a pyrolysis step comprising pyrolysis unit (reactor) 110 for producing first off-gas stream 103 (pyrolysis off-gas) and solid carbon stream (char stream) 105. An optional first liquid oil stream (first pyrolysis oil stream) may be withdrawn (not shown) from the pyrolysis step. While by the present invention a first liquid oil may optionally be generated in the pyrolysis step, the present embodiment of FIG. 1 describes the option of reducing as much as possible the generation of this first liquid oil, and instead keeping everything in the gas phase until after the HDO step (in HDO unit 112). The char stream 105 is conducted to a combustion/burning step in combustion unit/burner 114 utilizing oxygen 125′ produced by downstream electrolysis, thereby producing CO₂ stream 107 which is then fed (not shown) to a downstream methanation step. The pyrolysis off-gas 103 is conducted to a HDO step in HDO unit 112 for producing HDO-treated stream 109 and which is subsequently conducted to a separation step in separator 116. From separator 116 a water stream 115 is withdrawn, as so is a second liquid oil stream 113 and upgraded first off-gas stream 111. The latter is conducted to an olefin removal step comprising olefin removal reactor 118, e.g. olefin hydrogenation reactor, thereby generating a further upgraded first off-gas stream 117 which is free of olefins. This stream 117 is then conducted to methanation step comprising methanation reactor 120, thereby producing methane product (SNG) 119 and steam stream 121. Steam 121 and optionally also import steam 121′ are conducted to an electrolysis step comprising electrolysis unit (SOEC unit) 122 thereby producing oxygen stream 125 and hydrogen stream 123. A portion 125′ of the oxygen stream is used for char combustion as described above, while a portion of the hydrogen stream 123 is conducted as hydrogen stream 123′ to the HDO unit 112, as hydrogen stream 123″ to olefin hydrogenation reactor 118, and as hydrogen stream 123′″ to methanation reactor 120.

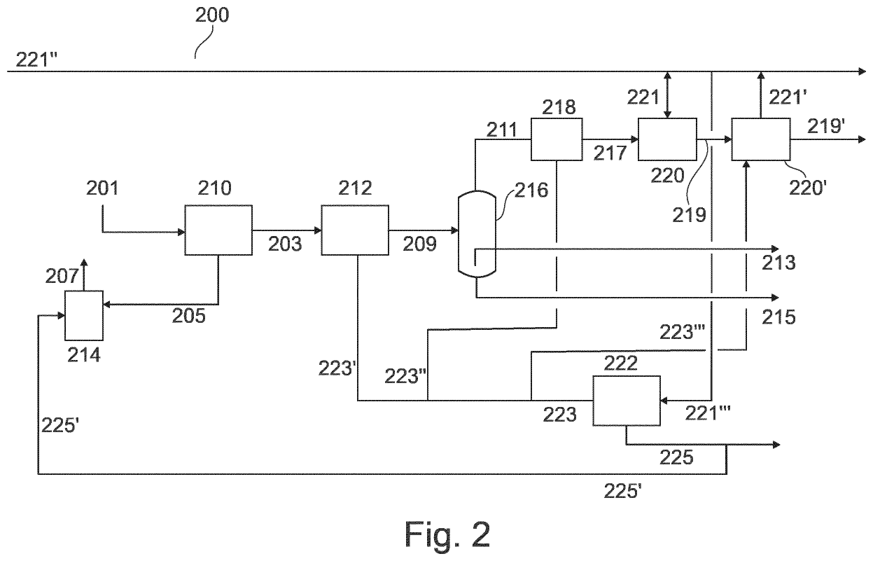

With reference to FIG. 2, a process/plant layout for producing methanol is shown. A solid renewable feedstock 201 is conducted to a pyrolysis step comprising pyrolysis unit (reactor) 210 for producing first off-gas stream 203 (pyrolysis off-gas) and solid carbon stream (char stream) 205. An optional first liquid oil stream (first pyrolysis oil stream) may be withdrawn (not shown) from the pyrolysis step. As in the embodiment of FIG. 1, the embodiment of FIG. 2 describes the option of reducing as much as possible the generation of this first liquid oil, and instead keeping everything in the gas phase until after the HDO step (in HDO unit 212). The char stream 205 is conducted to a combustion/burning step in combustion unit/burner 214 utilizing oxygen 225′ produced by downstream electrolysis, thereby producing CO₂ stream 207 which is optionally fed (not shown) to a downstream methanol synthesis, such as in instances where the CO₂ to CO molar ratio in the methanol synthesis gas 219 is low. The pyrolysis off-gas 203 is conducted to a HDO step in HDO unit 212 for producing HDO-treated stream 209 and which is subsequently conducted to a separation step in separator 216. From separator 216 a water stream 215 is withdrawn, as so is a second liquid oil stream 213 and upgraded first off-gas stream 211. The latter is conducted to an olefin removal step comprising olefin hydrogenation reactor 218, thereby generating a further upgraded first off-gas stream 217 which is free of olefins. This stream 217 is then conducted to a reforming unit such as a steam methane reforming (SMR) in an e-reformer 220 thereby producing a methanol synthesis gas 219 which is subsequently conducted to a methanol synthesis step in a methanol synthesis unit comprising methanol converter (methanol reactor) 220′. A raw methanol product 219′ is generated which is suitably further treated in a distillation column (not shown) for removing its water content, thereby producing the methanol product. During steam reforming and methanol synthesis, steam is generated 221, 221′. Some steam may also be imported 221″ as depicted in the figure. Part of the steam 221′″ used or generated in the process, is conducted to an electrolysis step comprising SOEC unit 222 thereby producing oxygen stream 225 and hydrogen stream 223. A portion 225′ of the oxygen stream is used for char combustion as described above, while a portion of the hydrogen stream 223 is conducted as hydrogen stream 223′ to the HDO unit 212, as hydrogen stream 223″ to olefin hydrogenation reactor 218, and as hydrogen stream 223′″ to methanol reactor 220.

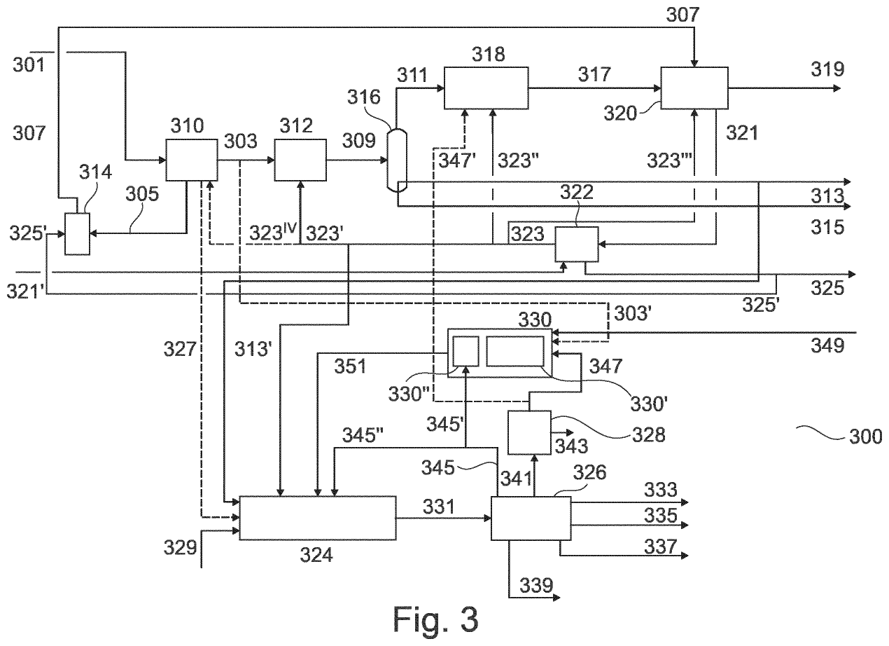

Now with reference to FIG. 3, a process for producing methane (SNG) as in FIG. 1, is further integrated with a refinery process. Hence, a solid renewable feedstock 301 is conducted to a pyrolysis step comprising pyrolysis unit (reactor) 310 for producing first off-gas stream 303 (pyrolysis off-gas), a solid carbon stream (char stream) 305, and an optional first liquid oil stream (first pyrolysis oil stream) 327. The char stream 305 is conducted to a combustion/burning step in combustion unit/burner 314 utilizing oxygen 325′ produced by downstream electrolysis, thereby producing CO₂ stream 307 which is then fed to downstream methanation reactor 320. The pyrolysis off-gas 303 is conducted to a HDO step in HDO unit 312 for producing HDO-treated stream 309 and which is subsequently conducted to a separation step in separator 316. From separator 316 a water stream 315 is withdrawn, as so is a second liquid oil stream 313 and upgraded first off-gas stream 311. The latter is conducted to an olefin removal step comprising olefin hydrogenation reactor 318, thereby generating a further upgraded first off-gas stream 317 which is free of olefins. This stream 317 is then conducted to methanation step comprising methanation reactor 320, thereby producing methane (SNG) 319 and steam stream 321. Steam 321 and optionally also import steam 321′ are conducted to an electrolysis step comprising SOEC unit 322 thereby producing oxygen stream 325 and hydrogen stream 323. A portion 325′ of the oxygen stream is used for char combustion as described above, while a portion of the hydrogen stream 323 is conducted as hydrogen stream 323′ to the HDO unit 312, as hydrogen stream 323″ to olefin hydrogenation reactor 318, and as hydrogen stream 323′″ to methanation reactor 320. Yet another portion of the hydrogen stream 323 is conducted as hydrogen stream 323′ to the pyrolysis unit 310 where for instance catalytic hydropyrolysis is conducted therein.

The second liquid oil stream 313 or a portion thereof 313′ is fed to a hydroprocessing step 324 (hydroprocessing section) for producing a main hydroprocessed stream 331. The first liquid oil stream 327 is suitably also conducted to the hydroprocessing step. The latter comprises the use of a catalytic unit for liquid oil stabilization to provide a stabilized liquid oil, as well as downstream HDO-unit and HDW/HDI unit (not shown). Hydrogen produced downstream such as make-up hydrogen stream 351 from a hydrogen producing unit (HPU 330) or hydrogen-rich stream 345″ from separation step (separation section) 326, are added to the above catalytic unit(s) in the hydroprocessing section. In the process line (conduit) supplying the hydrogen-rich stream 345″, there is no provision of a separation unit for removing H₂S and/or CO₂ optionally also for removing NH₃ and/or CO, from the hydrogen-rich stream.

The main hydrotreated stream 331 is conducted to said separation step 336 for thereby producing a water stream 339 as well as valuable products in the transportation fuel range, namely a naphtha stream 333, jet fuel stream 335 and diesel stream 337. Maritime (marine) fuel as a heavy fuel oil may also be separated (not shown). In addition, hydrogen-rich steam 345 is produced of which a portion 345′″ is recycled to the hydrogen processing step as described above, while another portion is suitably added to the HPU 330, particularly to a pressure swing adsorption unit (PSA-unit) 330″ which is used as hydrogen purification unit in the HPU: A second off-gas stream 341 comprising hydrocarbons is also produced in separation step 326. H₂S in stream 341 is removed as stream 343 in amine absorption unit 328 and the resulting stream 347 is then fed to HPU 330, optionally also as stream 347′ to olefin hydrogenation reactor 318 for thereby enabling production of valuable products, here methane (SNG). A portion 323v of the hydrogen stream from SOEC unit 322 is conducted to the hydroprocessing 324 as depicted in the figure, thereby bringing further integration and flexibility in the process (overall process/plant).

The use of the second off-gas stream 347 after removing H₂S therein in the HPU reduces significantly the use of an external hydrocarbon source such as natural gas stream 349 as hydrocarbon feed gas to the HPU 330. The need for natural gas stream 349 may be further reduced where a portion 303′ of the first pyrolysis off-gas is optionally also added to the HPU. The HPU comprises the use of, a catalytic steam reforming, suitably conducted in e-reformer 330′ and downstream PSA unit 330″ for hydrogen purification. The HPU produces make-up hydrogen stream 351 which is used in the hydroprocessing step 324. The use of external sources of hydrogen, e.g. imported hydrogen from outside battery limits, is thereby reduced or eliminated.

### EXAMPLES

### Example 1

Example 1 is in accordance with the process of FIG. 1. In this example a solid renewable feedstock 101 (straw or wood), is converted into a mixture of gas, bio-oil, and char by simple fast pyrolysis and the vapor i.e. first off-gas stream 103 (gas and bio-oil) is hydrotreated in the separate downstream HDO unit 112. The gas and liquid are then separated in the separator 116, as an upgraded first off-gas 111 and the so-called second liquid oil stream 113, respectively. The upgraded first off-gas stream 11 is less than 80 wt % of the solid renewable feedstock 101. A water stream 115 is also withdrawn. The further upgraded first off-gas 117 is then converted into methane 119 in methanation reactor 120. Table 1 shows the content of the streams. Without the olefin removal and subsequent methanation, the upgraded first gas stream 111 mainly consists of CO (about 27 mole %), CO₂ (about 20 mole %), CH₄ (about 46 mole %), and C2-C4 paraffins (C2+ saturated, about 3 mole %), as well as olefins (C2+ olefins, about 3 mole %). After the methanation the product gas stream 119 contains about 98 mole % CH₄. Hence, all carbon in the solid renewable feedstock, apart from the solid carbon stream (char) withdrawn in the pyrolysis step, is converted into the valuable product methane e.g. as substitute natural gas (SNG), and the valuable liquid oil stream 113 from which hydrocarbon products may be derived. Of the first off-gas stream 103 (pyrolysis off-gas fed to HDO unit 112) a minor portion of the carbon, about 30 wt % ends up in the methane stream 119 and about 70% in the second liquid oil stream 113. Thus, there is a carbon recovery of about 70 wt % in the liquid phase with respect to the first off-gas stream 103. This represents the carbon recovery over the HDO unit excluding the pyrolysis reactor.

TABLE 1
METHANATION
Total Stream Composition

| | | | | |
|---|---|---|---|---|
| Component | | | | |
| Name | wt % | kg/h | mole % | kmol/h |

Stream no. 103

| | | |
|---|---|---|
| Total carbon as | 46216 | 3848 |
| atomic C | | |
| Total oxygen as | 28241 | 1765 |
| atomic O | | |
| Total Hydrogen | 7546 | 7486 |
| as atomic H | | |

Stream no. 109

| | | | | |
|---|---|---|---|---|
| Aromatics | 22.57 | 18510 | 8.89 | 190 |
| C2+ olefins | 7.73 | 6342 | 3.98 | 85 |
| C2+ saturated | 8.85 | 7263 | 4.44 | 95 |
| Carbon Dioxide | 12.90 | 10584 | 11.27 | 240 |
| Carbon | 9.42 | 7725 | 12.92 | 276 |
| Monoxide | | | | |
| Hydrogen | 0.03 | 22 | 0.52 | 11 |
| Methane | 9.45 | 7752 | 22.64 | 483 |
| Oxygenates | 15.53 | 12739 | 6.51 | 139 |
| Water | 13.52 | 11090 | 28.84 | 616 |
| Total carbon as | | 46216 | | 3848 |
| atomic C | | | | |
| Total oxygen as | | 28240 | | 1765 |
| atomic O | | | | |
| Total Hydrogen | | 7567 | | 7508 |
| as atomic H | | | | |

Stream no. 111

| | | | | |
|---|---|---|---|---|
| Aromaticss | 0.09 | 24 | 0.02 | 0 |
| C2+ olefins | 4.00 | 1060 | 2.75 | 28 |
| C2+ saturated | 4.24 | 1123 | 2.70 | 27 |
| Carbon Dioxide | 34.18 | 9061 | 20.26 | 206 |
| Carbon | 28.84 | 7646 | 26.87 | 273 |
| Monoxide | | | | |
| Hydrogen | 0.08 | 22 | 1.08 | 11 |
| Methane | 28.30 | 7502 | 46.03 | 468 |
| Oxygenates | 0.11 | 29 | 0.05 | 1 |
| Water | 0.16 | 44 | 0.24 | 2 |
| Total carbon as | | 13230 | | 1101 |
| atomic C | | | | |
| Total oxygen as | | 11008 | | 688 |
| atomic O | | | | |
| Total Hydrogen | | 2273 | | 2255 |
| as atomic H | | | | |

Stream no. 113

| | | | | |
|---|---|---|---|---|
| Aromaticss | 41.57 | 18486 | 37.49 | 190 |
| C2+ olefins | 11.88 | 5282 | 11.27 | 57 |
| C2+ saturated | 13.81 | 6140 | 13.35 | 67 |
| Carbon Dioxide | 3.43 | 1523 | 6.85 | 35 |
| Carbon | 0.18 | 79 | 0.56 | 3 |
| Monoxide | | | | |
| Hydrogen | 2.2 ppmw | 0 | 97 ppm | 0 |
| Methane | 0.56 | 250 | 3.08 | 16 |
| Oxygenates | 28.58 | 12710 | 27.40 | 139 |
| Total carbon as | | 32987 | | 2746 |
| atomic C | | | | |
| Total oxygen as | | 7422 | | 464 |
| atomic O | | | | |
| Total Hydrogen | | 4059 | | 4027 |
| as atomic H | | | | |

Stream no. 117

| | | | | |
|---|---|---|---|---|
| Aromaticss | 0.09 | 24 | 0.02 | 0 |
| C2+ olefins | 0.05 | 13 | 99 ppm | 0 |
| C2+ saturated | 8.40 | 2233 | 5.45 | 55 |
| Carbon Dioxide | 34.10 | 9061 | 20.28 | 206 |
| Carbon | 28.78 | 7646 | 26.89 | 273 |
| Monoxide | | | | |
| Hydrogen | 0.08 | 20 | 0.99 | 10 |
| Methane | 28.23 | 7502 | 46.07 | 468 |
| Oxygenates | 0.11 | 29 | 0.05 | 1 |
| Water | 0.16 | 44 | 0.24 | 2 |
| Total carbon as | | 13230 | | 1101 |
| atomic C | | | | |
| Total oxygen as | | 11008 | | 688 |
| atomic O | | | | |
| Total Hydrogen | | 2333 | | 2315 |
| as atomic H | | | | |

Stream no. 119

| | | | | |
|---|---|---|---|---|
| Carbon Dioxide | 5 ppmw | 0 | 1.8 ppm | 0 |
| Carbon | ppmw | 0 | ppm | 0 |
| Monoxide | | | | |
| Hydrogen | 0.19 | 33 | 1.47 | 17 |
| Methane | 99.54 | 17665 | 98.28 | 1101 |
| Water | 0.28 | 49 | 0.24 | 3 |
| Total carbon as | | 13225 | | 1101 |
| atomic C | | | | |
| Total oxygen as | | 44 | | 3 |
| atomic O | | | | |
| Total Hydrogen | | 4478 | | 4443 |
| as atomic H | | | | |

Stream no. 125

| | | | | |
|---|---|---|---|---|
| Argon | 0.79 | 873 | 0.59 | 22 |
| Carbon Dioxide | 0.03 | 31 | 0.02 | 1 |
| Nitrogen | 45.86 | 50814 | 49.21 | 1814 |
| Oxygen | 53.22 | 58969 | 50.00 | 1843 |
| Water | 0.11 | 119 | 0.18 | 7 |

### Example 2

Similarly, in accordance with FIG. 2, when producing methanol, conducting HDO in the first off-gas stream 203 results in a carbon recovery of 71% (wt. basis) in the liquid phase—i.e. with respect to the first off-gas stream 103 —. This represents the carbon recovery over the HDO unit excluding the pyrolysis reactor. The solid carbon stream from pyrolysis (char) is optionally converted to CO₂ by combustion with oxygen produced by electrolysis, thus the CO₂ being lean in nitrogen. By adding the steam reforming and methanol synthesis step, there is nearly 100% carbon recovery, i.e. over the HDO and steam reforming/methanol synthesis step. In Table 2 the addition of CO₂ from char combustion is not included.

All carbon in the solid renewable feedstock, optionally including from the char withdrawn in the pyrolysis step by using the CO₂ 207 generated from the combustion of the char 205, is converted into the valuable product methanol 219′, as well as the valuable liquid oil stream 213 from which hydrocarbon products may be derived. The methanol may be further converted to gasoline thus further increasing the value. Table 2 shows the content of the streams.

TABLE 2
METHANOL
Total Stream Composition

| | | | | |
|---|---|---|---|---|
| Component | | | | |
| Name | wt % | kg/h | mole % | kmol/h |

Stream no. 203

| | | |
|---|---|---|
| Total carbon as | 46216 | 3848 |
| atomic C | | |
| Total oxygen as | 28241 | 1765 |
| atomic O | | |
| Total Hydrogen | 7546 | 7486 |
| as atomic H | | |

Stream no. 209

| | | | | |
|---|---|---|---|---|
| Aromates | 22.57 | 18510 | 8.89 | 190 |
| C2+ olefins | 7.73 | 6342 | 3.98 | 85 |
| C2+ saturated | 8.85 | 7263 | 4.44 | 95 |
| Carbon Dioxide | 12.90 | 10584 | 11.27 | 240 |
| Carbon | 9.42 | 7725 | 12.92 | 276 |
| Monoxide | | | | |
| Hydrogen | 0.03 | 22 | 0.52 | 11 |
| Methane | 9.45 | 7752 | 22.64 | 483 |
| Oxygenates | 15.53 | 12739 | 6.51 | 139 |
| Water | 13.52 | 11090 | 28.84 | 616 |
| Total carbon as | | 46216 | | 3848 |
| atomic C | | | | |
| Total oxygen as | | 28240 | | 1765 |
| atomic O | | | | |
| Total Hydrogen | | 7567 | | 7508 |
| as atomic H | | | | |

Stream no. 211

| | | | | |
|---|---|---|---|---|
| Aromates | 0.09 | 24 | 0.02 | 0 |
| C2+ olefins | 4.00 | 1060 | 2.75 | 28 |
| C2+ saturated | 4.24 | 1123 | 2.70 | 27 |
| Carbon Dioxide | 34.18 | 9061 | 20.26 | 206 |
| Carbon | 28.84 | 7646 | 26.87 | 273 |
| Monoxide | | | | |
| Hydrogen | 0.08 | 22 | 1.08 | 11 |
| Methane | 28.30 | 7502 | 46.03 | 468 |
| Oxygenates | 0.11 | 29 | 0.05 | 1 |
| Water | 0.16 | 44 | 0.24 | 2 |
| Total carbon as | | 13230 | | 1101 |
| atomic C | | | | |
| Total oxygen as | | 11008 | | 688 |
| atomic O | | | | |
| Total Hydrogen | | 2273 | | 2255 |
| as atomic H | | | | |

Stream no. 213

| | | | | |
|---|---|---|---|---|
| Aromates | 41.57 | 18486 | 37.49 | 190 |
| C2+ olefins | 11.88 | 5282 | 11.27 | 57 |
| C2+ saturated | 13.81 | 6140 | 13.35 | 67 |
| Carbon Dioxide | 3.43 | 1523 | 6.85 | 35 |
| Carbon | 0.18 | 79 | 0.56 | 3 |
| Monoxide | | | | |
| Hydrogen | 2.2 ppmw | 0 | 97 ppm | 0 |
| Methane | 0.56 | 250 | 3.08 | 16 |
| Oxygenates | 28.58 | 12710 | 27.40 | 139 |
| Total carbon as | | 32987 | | 2746 |
| atomic C | | | | |
| Total oxygen as | | 7422 | | 464 |
| atomic O | | | | |
| Total Hydrogen | | 4059 | | 4027 |
| as atomic H | | | | |

Stream no. 217

| | | | | |
|---|---|---|---|---|
| Aromates | 0.09 | 24 | 0.02 | 0 |
| C2+ olefins | 3.99 | 1060 | 2.67 | 28 |
| C2+ saturated | 4.23 | 1123 | 2.62 | 27 |
| Carbon Dioxide | 34.10 | 9061 | 19.68 | 206 |
| Carbon | 28.78 | 7646 | 26.10 | 273 |
| Monoxide | | | | |
| Hydrogen | 0.31 | 82 | 3.91 | 41 |
| Methane | 28.23 | 7502 | 44.71 | 468 |
| Oxygenates | 0.11 | 29 | 0.05 | 1 |
| Water | 0.16 | 44 | 0.23 | 2 |
| Total carbon as | | 13230 | | 1101 |
| atomic C | | | | |
| Total oxygen as | | 11008 | | 688 |
| atomic O | | | | |
| Total Hydrogen | | 2333 | | 2315 |
| as atomic H | | | | |

Stream no. 219′

| | | | | |
|---|---|---|---|---|
| Methanol | 100.00 | 35239 | 100.00 | 1100 |
| Total carbon as | | 13209 | | 1100 |
| atomic C | | | | |
| Total oxygen as | | 17595 | | 1100 |
| atomic O | | | | |
| Total Hydrogen | | 4434 | | 4399 |
| as atomic H | | | | |

Stream no. 225

| | | | | |
|---|---|---|---|---|
| Argon | 0.79 | 513 | 0.59 | 13 |
| Carbon Dioxide | 0.03 | 18 | 0.02 | 0 |
| Nitrogen | 45.86 | 29869 | 49.21 | 1066 |
| Oxygen | 53.22 | 34666 | 50.00 | 1083 |
| Water | 0.11 | 70 | 0.18 | 4 |

1. A process for producing methane or methanol, said process comprising the steps of:
i) conducting a solid renewable feedstock to a thermal decomposition step for producing: a first off-gas stream comprising hydrocarbons, and a solid carbon stream; wherein the thermal decomposition step is a pyrolysis step or a hydrothermal liquefaction step;
upgrading the first off-gas stream by conducting it to a hydro/deoxygenation (HDO/DO) step, in which said HDO/DO step is conducted in the absence of steam, and a subsequent separation step for generating water, a second liquid oil stream and an upgraded first off-gas stream;
ii) conducting the upgraded first off-gas stream to an olefin removal step, for generating a further upgraded first off-gas stream which is free of olefins;
iii-1) conducting the further upgraded first off-gas stream to a methanation step under the generation of steam for producing said methane;
or
iii-2) conducting the further upgraded first off-gas stream to a steam reforming step for producing a methanol synthesis gas and subsequently conducting the methanol synthesis gas to a methanol synthesis step under the generation of steam for producing said methanol;
iv) conducting steam, to an electrolysis step for producing an oxygen stream and a hydrogen stream;
v) conducting at least a portion of the hydrogen stream from the electrolysis step to any of the: thermal decomposition step including the HDO-step, olefin hydrogenation step, methanation step, methanol synthesis step, or combinations thereof.

2. The process according to claim 1, further comprising:
i-1) combusting at least a portion of the solid carbon stream with at least a portion of the oxygen stream from the electrolysis, for generating heat and a carbon dioxide stream; and conducting at least a portion of the carbon dioxide stream to step iii-1) or iii-2);
or
i-2) conducting at least a portion of the solid carbon stream to a gasification step with steam and/or oxygen produced in the process.

3. The process according to claim 1, further comprising converting the methanol into gasoline.

4. The process according to claim 1, wherein the upgraded first off-gas stream is less than 80 wt % of said solid renewable feedstock.

5. The process according to claim 1, wherein: of the first off-gas stream, 40 wt % or less ends up in the methane or methanol; and 60 wt % or more ends up in the second liquid oil stream.

6. The process according to claim 1, wherein the pyrolysis step is:
a fast pyrolysis step, in which the vapor residence time is 10 seconds or less and is selected from: simple fast pyrolysis; in-situ catalytic fast pyrolysis (in-situ CFP); ex-situ catalytic fast pyrolysis (ex-situ CFP); reactive catalytic fast pyrolysis (RCFP);
hydropyrolysis (HP);
catalytic fast hydropyrolysis (CHP); or
intermediate pyrolysis, in which the vapor residence time is in the range 10 seconds-5 minutes; or slow pyrolysis, in which the solid residence time is in the range 5 minutes-2 hours.

7. The process according to claim 1, wherein the solid renewable feedstock is:
a lignocellulosic biomass including: wood products, forestry waste, and agricultural residue; and/or
municipal waste, in particular the organic portion thereof.

8. The process according to claim 1, wherein the steam reforming step in step iii-2 is conducted in an electrically heated reformer (e-reformer).

9. The process according to claim 1, wherein between step i) and ii) or between step ii) and iii), said first off-gas stream or said upgraded first off-gas stream or said further upgraded first off-gas stream, passes to a separation unit for sulfur removal.

10. The process according to claim 1, further comprising:
vi) conducting the second liquid oil stream of step i) to a hydroprocessing step for producing a main hydroprocessed stream;
vii) conducting the main hydroprocessed stream to a separation step for producing:
an aqueous stream,
a hydrogen-rich stream,
a second off-gas stream comprising hydrocarbons,
and a hydrocarbon product, boiling at above 50° C.;
viii) conducting the second off-gas stream from step vii) to said olefin removal step i.e. step ii), or to said step iii), and/or to a hydrogen producing unit (HPU) for producing a make-up hydrogen stream;
the process further comprising:
conducting the hydrogen-rich stream from step vii), or a portion thereof, to the hydroprocessing step, without subjecting said hydrogen-rich stream, or the portion thereof, to a separation step for removing H₂S and/or CO₂.

11. The process according to claim 10, wherein prior to conducting step viii), said second off-gas stream from step vii) passes to a separation unit, the separation unit preferably being at least one of an amine absorption unit, a caustic scrubber, and a sulfur absorbent unit, for removing H₂S

12. The process according to claim 10, wherein the HPU comprises subjecting the second off-gas stream to: cleaning in a cleaning unit.

13. The process according to claim 12, further comprising:
ix) conducting a portion of the first off-gas stream of step i) to the HPU;
and/or
x) producing in step iii-2) a methanol synthesis purge gas, suitably comprising CH₄, H₂, CO, CO₂, CH₃OH and inerts such as N₂, and conducting at least a portion of the methanol synthesis purge gas to the HPU;
and/or
xi) further producing in the separation step vii) an LPG stream and feeding the LPG stream to the HPU.

14. The process according to claim 10, wherein in step vi) the hydroprocessing step comprises:
vi-1) conducting said second liquid oil stream and/or said first liquid oil stream, suitably a combination thereof, to a first catalytic hydrotreating unit under the addition of hydrogen for producing a first hydrotreated stream,
vi-2) conducting the first hydrotreated stream to a dewaxing step comprising a second catalytic hydrotreating unit under the addition of hydrogen for producing said main hydrotreated stream;
wherein prior to conducting step vi-1), said second liquid oil stream and/or said first liquid oil stream is conducted to a catalytic unit for liquid oil stabilization under the addition of hydrogen.

15. The process according to claim 10, comprising:
conducting at least a portion of the hydrogen stream from the electrolysis step to the hydroprocessing step vi), suitably to said catalytic unit for liquid oil stabilization.

16. The process according to claim 14, wherein between step vi-1) and vi-2) the process further comprises conducting the first hydrotreated stream to a separator for removing H₂S, NH₃, and H₂O, thereby producing said first hydrotreated stream.

17. The process according to claim wherein the separation step vii) comprises:
vii-1) conducting the main hydrotreated stream to a separator for producing said aqueous stream, said hydrogen-rich stream, and a heavy hydrocarbon stream;
vii-2) conducting the heavy hydrocarbon stream to a fractionation step for producing said second off-gas stream and said hydrocarbon product.

18. The process according to claim 10, wherein step vi) further comprises co-feeding to the hydroprocessing step:
a first generation renewable feedstock selected from the group of renewable feeds which is already liquid; and/or
a second generation renewable feedstock selected from the group of waste oil and fats: such as cooking oil, animal fats and crude tall oil (CTO); and/or
a feedstock comprising any of refused derived fuel (RDF) including municipal solid waste, plastic derived oil, and tire pyrolysis oil; and/or
a feedstock of fossil fuel origin, such as diesel, kerosene, naphtha, and vacuum gas oil (VGO), and/or recycling a hydrocarbon product.

19. A plant carrying out the process according to claim 1, comprising:
a pyrolysis unit or hydrothermal liquefaction unit, arranged to receive a solid renewable feedstock and provide a first off-gas stream, and a solid carbon stream;
a HDO/DO unit, arranged to receive, in the absence of steam, the first off-gas stream, and provide a HDO/DO treated stream; and a downstream separator arranged to receive the HDO/DO treated stream and provide a water stream, a second liquid oil stream, and an upgraded first off-gas stream;
an olefin removal reactor arranged to receive the upgraded first off-gas and provide a further upgraded first off-gas stream which is free of olefins;
a methanation reactor arranged to receive the further upgraded first off-gas stream and provide, under the generation of steam, a methane product stream;
or
a reforming unit arranged to receive the further upgraded first off-gas stream and provide a methanol synthesis gas, and a downstream methanol synthesis unit arranged to receive the methanol synthesis gas and to provide, under the generation of steam, a methanol product stream;
an electrolysis unit arranged to receive at least a portion of the steam generated in the methanation reactor or reforming unit or methanol synthesis unit, and provide an oxygen stream and hydrogen stream;
a conduit for supplying at least a portion of the hydrogen stream from the electrolysis unit to any of the: pyrolysis unit or hydrothermal liquefaction unit, the HDO unit, olefin removal reactor, methanation reactor, methanol synthesis units, or combinations thereof.

20. The plant according to claim 19, further comprising:
a hydroprocessing section arranged to receive the second liquid oil stream and provide a main hydroprocessed stream;
a separation section arranged to receive the main hydroprocessed stream and provide: an aqueous stream, a hydrogen-rich stream, a second off-gas stream comprising hydrocarbons, and a hydrocarbon products;
a conduit for supplying the second off-gas stream from step vii) to said olefin removal reactor; and/or to a hydrogen producing unit (HPU) to provide a make-up hydrogen stream;
a conduit for conducting the hydrogen-rich stream or a portion thereof, to the hydroprocessing section, without providing a separation unit therein for removing H₂S and/or CO₂.

21. The plant according to claim 20, wherein the hydroprocessing section comprises:
a catalytic unit for liquid oil stabilization under the addition of hydrogen, said catalytic unit being arranged to receive said second liquid oil stream and/or said first liquid oil stream, and provide a stabilized liquid oil;
a first catalytic hydrotreating unit arranged to receive the stabilized liquid oil and provide a first hydrotreated stream.

22. The process of claim 1, wherein the step i) conducting a solid renewable feedstock to a thermal decomposition step produces a first liquid oil stream.

23. The process of claim 1, wherein the hydro/deoxygenation (HDO/DO) step is a hydrodeoxygenation (HDO) step, a deoxygenation (DO) step or a hydrodeoxygenation (HDO) and deoxygenation (DO) step

24. The process of claim 1, wherein the step iv) comprises conducting at least a portion of the steam generated in step iii-1) or iii-2) to the electrolysis step.

25. The process of claim 1, wherein the step i-2) comprises conducting at least a portion of the solid carbon stream to the gasification step with at least a portion of the oxygen stream from electrolysis for producing a synthesis gas stream and conducting it to step iii-1) or iii-2).
